SEQUENCE LISTING (54 sequences)

SEQ ID NO: 1 - protein, 499 aa, Shewanella halifaxensis
MNNDNLSGTPEHIIDQVKINVIETENYKVAPVTTPQDIKSYGWNQTCGIPVLSEEDKTSFTFNFTAPELHEDQQYCFEFNATTSKNTKYTTKTTINVVAPTLELYIDNASLPTLHQLMHIIESYEENLTRTRFISWGRVSITDEQVRDMLNISTFPLVSNNTSQKLVDAVKSYAQSKNRLNIEIYSNTTHALKNIKPIISSLSGNPNVNIAEINLYDDGSAEYINLYNWKNTPNKIDALNADLLVMKDYVEGYSTQSPSYMSSRYNWHKLYDTEYHFLRADYLTIEPNLNDLRDYLGNSLEQMDWGKFEQLSKAKQQLFLSIVGFDKDSLEKSYANSPNKNFVFTGTTTWAGNETREFYAKQQINVINNAINETSPLYLGEEYDLFFKGHPRGGDINNMILNAFKDMINIPASISFEVLMMTGSLPDKVAGIASSLYFTIPAEKVDFIVFTSSDDITDREEALKSPLVQVMMKLGIVEKQAVQFWSDLPNCESGVCINN

SEQ ID NO: 2 - protein, 296 aa, Helicobacter acinonychis
MGTIKKPLIIAGNGPSIKDLDYALFPKDFDVFRCNQFYFEDKYYLGREIKGVFFNPCVLSPQMQTAQYLMDNGEYSIERFFCSVSTDRHDFDGDYQTILPVDGYLKAHYPFVCDTFSLFKGHEEILKHVKYHLKTYSKELSAGVLMLLSAVVLGYKEIYLVGIDFGASSWGHFYDEHQSQHFSNHMADCHNIYYDMLTICLCQKYAKLYALAPNSPLSHLLTLNPQAKYPFELLDKPIGYTSDLIISSPLEEKLLEFKNIEEKLLEFKNIEEKLLASRLNNILRKIKRKILPFFGG

SEQ ID NO: 3 - protein, 495 aa, Vibrio alginolyticus
MSDDDHQTHNNSVKFDVIDNQEFTLNPIQNDSNPDLESFSWTQTCGSISIIPSDTSQTVNLTLTAPKLDNDEEYCFQFKGTSKNGDQYSTDAKVSVVSPSLEVYVDHASLPSLHQVLDIIASAEAHPTAERYVSWGRINPTQEHLAQLNISRFPLESNHTSAEMLEAISAFAEDHHRLKVSISTNTLKSYDNLKLMLQRLHKQPHVDIDNIRLYDDGSAEYVNLYNWRNTQDKTYLLQQAGDNLKNIILGSGGSSAPWLTTQFNWHSLYPTEYSMLRSDFLTLDPKLHELKEYLGDSLKQMQWDKYAKLSSEQQALFLEIVGFDQNWLQVEYDKSPLANFVFTGTTTWAGGEEKEFYAKQQVNIINNAINETSPYYIGKEHDLFFKGHPRGGVINDIIISSFDNMVNIPSAISFEVLMMTDMLPDTIAGVASSLYFTIPAENIKFIVFTSSEEITDREQALKSPLVQVMMTLGIVKEENVLFWADMPDCSSGTCI

SEQ ID NO: 4 - protein, 388 aa, Pasteurela multocida
MKTITLYLDPASLPALNQLMDFTQNNEDKTHPRIFGLSRFKIPDNIITQYQNIHFVELKDNRPTEALFTILDQYPGNIELNIHLNIAHSVQLIRPILAYRFKHLDRVSIQQLNLYDDGSMEYVDLEKEENKDISAEIKQAEKQLSHYLLTGKIKFDNPTIARYVWQSAFPVKYHFLSTDYFEKAEFLQPLKEYLAENYQKMDWTAYQQLTPEQQAFYLTLVGFNDEVKQSLEVQQAKFIFTGTTTWEGNTDVREYYAQQQLNLLNHFTQAEGDLFIGDHYKIYFKGHPRGGEINDYILNNAKNITNIPANISFEVLMMTGLLPDKVGGVASSLYFSLPKEKISHIIFTSNKQVKSKEDALNNPYVKVMRRLGIIDESQVIFWDSLKQL

SEQ ID NO: 5 - protein, 496 aa, Photobacterium leiognathi
MNDNQNTVDVVVSTVNDNVIENNTYQVKPIDTPTTFDSYSWIQTCGTPILKDDEKYSLSFDFVAPELDQDEKFCFEFTGDVDGKRYVTQTNLTVVAPTLEVYVDHASLPSLQQLMKIIQQKNEYSQNERFISWGRIGLTEDNAEKLNAHIYPLAGNNTSQELVDAVIDYADSKNRLNLELNTNTAHSFPNLAPILRIISSKSNILISNINLYDDGSAEYVNLYNWKDTEDKSVKLSDSFLVLKDYFNGISSEKPSGIYGRYNWHQLYNTSYYFLRKDYLTVEPQLHDLREYLGGSLKQMSWDGFSQLSKGDKELFLNIVGFDQEKLQQEYQQSELPNFVFTGTTTWAGGETKEYYAQQQVNVVNNAINETSPYYLGREHDLFFKGHPRGGIINDIILGSFNNMIDIPAKVSFEVLMMTGMLPDTVGGIASSLYFSIPAEKVSFIVFTSSDTITDREDALKSPLVQVMMTLGIVKEKDVLFWSDLPDCSSGVCIAQY

SEQ ID NO: 6 - protein, 498 aa, Photobacterium phosphoreum
MSEDYTPSIYKLDINQTVTDEENVNLEPTNQSNIIFTKHSWVQTCGTQQLLTTQNKESISLSIMAPRLEKDEKYCFDFNGVNNKGDEYTTKVILNVVSPSLEVYVDHASLPTLQQLMDIIKSEEQNPTTQRYISWGRIHPTIEQMKELNITSFVLGSNHTTSELVQAIVKQAQTKHRLNVKLSSNTAHSYYNLIPILKALNTFNNVTVTNIDLYDDGSAEYVNLYNWRNTENKIYNLQLGKASLEDVISGVTENFSGPAMASIYNWQQLYPTEYHFLRKDYLTLEPSLHELRDYLGDSLKQMQWDGFKTFDVKQKELFLSIVGFDKQKLQNEYNSSNLPNFVFTGTTVWAGNHEREYYAKQQINVINNAINESSPYYLGKSYDLFFKGHPGGGIINTLIMQNFPKMIDIPAKISFEVLMMTDMLPDAVAGMASSLYFTIPPDKIKFIVFTSSDTITDRETALQSPLVQVMIKLGIVKEENVLFWADLPNCETGICIAA

SEQ ID NO: 7 - protein, 482 aa, Photobacterium leiognathi
MNDNQNTVDVVVSTVNDNVIENNTYQVKPIDTPTTFDSYSWIQTCGTPILKDDEKYSLSFDFVAPELDQDEKFCFEFTGDVDGKRYVTQTNLTVVAPTLEVYVDHASLPSLQQLMKIIQQKNEYSQNERFISWGRIRLTEDNAEKLNAHIYPLAGNNTSQELVDAVIDYADSKNRLNLELNTNTGHSFRNIAPILRATSSKNNILISNINLYDDGSAEYVSLYNWKDTDNKSQKLSDSFLVLKDYLNGISSEKPNGIYSIYNWHQLYHSSYYFLRKDYLTVETKLHDLREYLGGSLKQMSWDTFSQLSKGDKELFLNIVGFDQEKLQQEYQQSELPNFVFTGTTTWAGGETKEYYAQQQVNVVNNAINETSPYYLGREHDLFFKGHPRGGIINDIILGSFNNMIDIPAKVSFEVLMMTGMLPDTVGGIASSLYFSIPAEKVSFIVFTSSDTITDREDALKSPLVQVMMTLGIVKEKDVLFWC

SEQ ID NO: 8 - protein, 493 aa, Photobacterium damselae
MDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYSNTAHSFNNLASIIQSLYNKDNVTISHVSLYDDGSAEYVNLYQWKDTPNKIEVLERDISLLDDYLAGTSPDTPKGMGNRYNWHKLYDTDYYFLREDYLDVEANLHDLRDYLGSSVKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWADLPDCSSGVCIDK

SEQ ID NO: 9 - protein, 392 aa, Pasteurella oralis
MSNWAKSVTIYLDPASLPALNQLMDFTQKSDDKETARIFGHTRFKMPEEIQKQYQHIDMVGIKDEKPTEELFSILDQYPESLELDLHMSIAHATKLIQPILAYRFKHPSRVSIRSLNLYDDGSLEYVGLENLQDVDIPKAIAQAEQQLASFLMTGKAKFDNPIVARYVWQSQFPVKYHFLSPEYFEKAAFIKPLKEYLQDNYQKMALFAYQDLSSEKQALYLKLVGFNDQIKQLLETTEKKFIFTGTTTWEAKTDVREYYAQQQLNLLKHFTQPNGELFIGDDYKVYFKGHPKGDEINEYILNNAKDIINIPANISFEILMMTGLLPDKVGGIASSLYFSLPKEKISHIIFTTNKQVKSKEDALNNPYVKVMQRLGIIDESQVIFWDTLKQL

SEQ ID NO: 10 - protein, 498 aa, Photobacterium kishitanii
MSEDYTPPIHKLDINQTVTDEENVKLEPTNQSNIIFTKHSWVQTCGTQQLLTTQNKESISLSIMAPRLEKDEKYCFDFNGVNDKGDKYITKVILNVVSPSLEVYVDHASLPALQQLMDIIKSEEQNPTTQRYISWWRINPTDEQMKELNITRFPLINNHTSSELVQAIVKQAQTKHRLNVKLSSNTARSFYNLMPILKALNTFNNVTITNIDLYDDGSAEYVDLYNWRNSVNKIYNLQLGKDSLEDVISGVTDNYSGSEIASIYNWQQLYPTKYHFLRKDYLTLEPSLHELRDYLGDSLKQMQWDGFKKFNSKQQELFLSIVGFDKQKLQNEYNSSNLPNFVFTGTTVWAGDHEKEYYANKQIDVIDNAINESSPYYLGKSYDLFFKGHPGAGIINTLIMQNFPTMIDIPSIISFEVLMMTDMLPDAVAGMASSLYFTIPSDKIKFIVFTSSDTITDRETALQSALVQVMIKLGIVKEENVLFWADLPNCETGICIAA

SEQ ID NO: 11 - protein, 660 aa, Photobacterium damsela
MNSDNTSLKETVSSNSADVVETETYQLTPIDAPSSFLSHSWEQTCGTPILNESDKQAISFDFVAPELKQDEKYCFTFKGITGDHRYITNTTLTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWADHKVNSMEVAIDEACTRIIAKRQPTASDLRLVIAIIKTITDLERIGDVAESIAKVALESFSNKQYNLLVSLESLGQHTVRMLHEVLDAFARMDVKAAIEVYQEDDRIDQEYESIVRQLMAHMMEDPSSIPNVMKVMWAARSIERVGDRCQNICEYIIYFVKGKDVRHTKPDDFGTMLD

SEQ ID NO: 12 - protein, 503 aa, Photobacterium sp.
MNNGDNSEGHEPSVYKMSINQTVTDEENVNLEPTNQSNVIFTKHSWVQTCGIQQLLTTQNKESISLSVIAPRLEKDEKYCFDFNGTNNKGDTYVTKIILNVVSPSLEVYVDHASLPTLQQLMDIIKSEEENPTTQRYIAWGRIHPTTEQMKELNITSFVLESNHTTSELVQAIVKQAQTKHRLNVKLSSNTAHSYYNLTPILKALNTFNNVTVTNIDLYDDGSAEYVNLYNWRNVENKIYNLQLGKDSLEDVISGVADDFSGSSMSSIYNWQQLYPTKYHFLRKDYLTLETSLHELRDYLGDSLKQMQWDGFKKFNIKQQELFLSIVGFDKQKLQNEYNSSNLPNFVFTGTTIWAGDHEREYYAQQQINVINNAINESSPYYLGKDYDLFFKGHPGGGIINTLIMQNFPTMIDIPSKISFEVLMMTDMLPDAVAGMASSLYFTIPSDKIQFIVFTSSDTITDRETALKSPLVQVMIKLGIVKEENVLFWADLPNCDTGICIAA

SEQ ID NO: 13 - DNA, 1500 bp, synthetic construct
  misc_feature 1..1500: note = Codon optimized DNA encoding the alpha-2,6-sialyltransferase Shal
  source 1..1500: mol_type = other DNA
atgaataatgataatctgagcggtacaccggaacatattattgatcaggtgaaaatcaacgtgatcgaaaccgagaactataaagttgcaccggttaccacaccgcaggatatcaaaagctatggttggaatcagacctgtggtattccggttctgagcgaagaagataaaaccagctttacctttaactttaccgcaccggaactgcatgaagatcagcagtattgctttgaatttaatgccaccacgagcaagaacaccaaatataccaccaaaaccaccattaatgttgttgcaccgacactggaactgtatattgataatgcaagcctgccgacactgcaccagctgatgcatattatcgaaagctacgaagaaaatctgacccgtacacgttttattagctggggtcgtgttagcattaccgatgaacaggttcgtgatatgctgaacattagcacctttccgctggttagcaataataccagccagaaactggttgatgccgttaaaagttatgcccagagcaaaaatcgtctgaacatcgaaatctatagcaataccacacatgccctgaaaaacatcaaaccgattattagcagcctgagcggcaatccgaatgttaatattgcagaaatcaacctgtatgatgatggcagcgcagaatacattaatctgtacaattggaaaaacaccccgaacaaaattgatgcactgaatgccgatctgctggtgatgaaagattatgttgaaggttatagcacccagagtccgagctatatgagcagccgttataattggcataaactgtatgataccgagtaccattttctgcgtgcagattatctgaccattgaaccgaatctgaatgatctgcgtgattatctgggtaatagcctggaacaaatggattggggtaaatttgaacagctgagcaaagcaaaacagcagctgttcctgagcattgtgggttttgataaagacagcctggaaaaaagctacgcaaatagcccgaataaaaactttgtttttaccggcaccaccacctgggcaggtaatgaaacccgtgaattttatgcgaaacagcagattaacgtgatcaacaacgccattaatgaaacgagtccgctgtatctgggtgaagaatatgacctgtttttcaaaggtcatccgcgtggtggtgatattaacaatatgattctgaacgcctttaaagacatgatcaatattccggcaagcatcagctttgaagttctgatgatgaccggtagcctgccggataaagtggcaggtattgcaagcagcctgtattttaccattccggcagaaaaagtggatttcattgtgtttaccagcagcgacgatattaccgatcgtgaagaagcactgaaatcaccgctggttcaggttatgatgaaactgggtattgttgagaaacaggcagttcagttttggagcgatctgccgaattgtgaaagcggtgtttgcattaacaactaa

SEQ ID NO: 14 - DNA, 891 bp, synthetic construct
  misc_feature 1..891: note = Codon optimized DNA encoding the alpha-2,6-sialyltransferaseHAC1268
  source 1..891: mol_type = other DNA
atgggcaccatcaaaaaaccgctgattattgcaggtaatggtccgagcattaaagatctggattatgcactgtttccgaaagattttgatgtgtttcgttgcaaccagttctatttcgaggataaatactatctgggtcgcgaaattaaaggcgtgttttttaacccgtgtgttctgagtccgcagatgcagaccgcacagtatctgatggataatggtgaatatagcatcgagcgttttttttgtagcgttagcaccgatcgtcatgatttcgatggtgattatcagaccattctgccggttgatggttatctgaaagcacattatccgtttgtgtgtgataccttcagcctgtttaaaggccatgaagaaattctgaaacacgtgaagtatcatctgaaaacctacagcaaagaactgagtgccggtgttctgatgctgctgagcgcagttgttctgggttataaagaaatttatctggtgggcattgattttggtgcaagcagctggggtcatttttatgatgaacatcagagccagcatttcagcaatcatatggcagattgccacaacatctattatgatatgctgaccatttgcctgtgccagaaatatgcaaaactgtatgcactggcaccgaatagtccgctgagccatctgctgaccctgaatccgcaggcaaaatatccttttgaactgctggataaaccgattggttataccagcgatctgattattagctcaccgctggaagaaaagctgctggaatttaagaacatcgaagagaaactgcttgagttcaaaaacatcgaggaaaaattgctggcaagccgtctgaataacattctgcgtaaaatcaaacgcaaaatcctgccgttttttggtggctaa

SEQ ID NO: 15 - DNA, 1488 bp, synthetic construct
  misc_feature 1..1488: note = Codon optimized DNA encoding the alpha-2,6-sialyltransferaseValg2
  source 1..1488: mol_type = other DNA
atgagtgatgatgaccatcagacccataacaatagcgtgaaatttgatgtgatcgacaaccaagaatttacgctgaacccgattcagaatgatagcaatccggatctggaaagctttagctggacccagacctgtggtagcattagcattattccgagcgataccagccagaccgttaatctgaccctgaccgcaccgaaactggataatgatgaagaatattgcttccagtttaagggcaccagcaaaaatggtgatcagtatagcaccgatgcaaaagttagcgttgttagcccgagcctggaagtttatgttgatcatgcaagcctgccgagcctgcatcaggttctggatattattgcaagcgcagaagcacatccgaccgcagaacgttatgttagctggggtcgtattaatccgacacaagaacatctggcacagctgaatattagccgttttccgctggaaagcaatcataccagcgcagaaatgctggaagccattagcgcatttgcagaagatcatcatcgtctgaaagtgagcattagtaccaatacgctgaaaagctatgacaacctgaaactgatgctgcagcgtctgcataaacagccgcatgttgatattgataacatccgcctgtatgatgatggtagcgcagaatatgtgaatctgtataattggcgtaacacccaggataaaacctatctgctgcaacaggcaggcgataatctgaaaaatatcattttaggtagcggtggtagcagcgcaccgtggctgaccacacagtttaattggcatagcctgtatccgaccgaatatagcatgctgcgtagcgattttctgaccttagatccgaaactgcatgagctgaaagaatatctgggtgatagcctgaaacaaatgcagtgggataaatatgcaaaactgagcagcgaacagcaggcactgtttctggaaattgttggttttgatcagaattggctgcaggtcgagtatgataaaagtccgctggcaaattttgtttttaccggcaccaccacctgggcaggcggtgaagaaaaagaattttatgccaaacagcaggtcaacatcattaacaacgccattaatgaaacgagcccgtattatatcggtaaagaacatgacctgttcttcaaaggtcatccgcgtggtggtgttattaacgatattatcattagcagcttcgataacatggtgaatattccgagtgccattagctttgaagttcttatgatgaccgatatgctgccggataccattgccggtgttgcaagcagcctgtattttaccattccggcagaaaacatcaagttcattgtgtttaccagcagcgaagaaattaccgatcgtgaacaggcactgaaatcaccgctggttcaggtaatgatgaccctgggtattgttaaagaagagaacgtactgttttgggcagatatgccggattgtagcagcggtacatgtatttaa

SEQ ID NO: 16 - DNA, 1167 bp, synthetic construct
  misc_feature 1..1167: note = Codon optimized DNA encoding the alpha-2,6-sialyltransferasePmult
  source 1..1167: mol_type = other DNA
atgaaaaccattacgctgtatttagatccggcaagcctgcctgcactgaaccagctgatggattttacccagaataacgaggataaaacccatccgcgtatctttggtctgagccgctttaaaatcccggataacattattacccagtaccagaacatccacttcgtggaactgaaagataatcgtccgaccgaagcactgtttaccattctggatcagtatccgggtaatatcgaactgaacatccatctgaatattgcccatagcgttcagctgattcgtccgattctggcatatcgttttaaacatctggatcgtgttagcattcagcagctgaacctgtatgatgatggtagcatggaatatgtggatctggaaaaagaagagaacaaagatatcagcgcagaaatcaaacaggcagaaaaacagctgagccattatctgctgaccggcaaaattaagtttgataatccgaccattgcgcgttatgtttggcagagcgcatttccggttaaatatcattttctgagcaccgactatttcgagaaagcagaatttctgcagccgctgaaagaatatctggcagaaaattaccagaaaatggattggaccgcatatcagcaactgacaccggaacagcaggcattttatctgaccctggttggttttaacgatgaagttaaacagagcctggaagttcagcaggccaaatttatctttaccggcaccaccacctgggaaggtaataccgatgttcgtgaatattatgcacagcaacagctgaatctgctgaatcattttacacaggccgaaggtgacctgtttattggtgatcattacaaaatctatttcaaaggtcatccgcgtggtggcgaaattaatgattatattctgaacaacgccaaaaacatcaccaacattccggcaaacattagctttgaagttctgatgatgaccggtctgctgccggataaagttggtggtgttgcaagcagcctgtattttagcctgccgaaagaaaaaatcagccacatcattttcaccagcaacaaacaggtgaaaagcaaagaagatgcactgaataacccgtacgttaaagttatgcgtcgtctgggtattattgatgaaagccaggttatcttttgggacagcctgaaacagctgtaa

SEQ ID NO: 17 - DNA, 1491 bp, synthetic construct
  misc_feature 1..1491: note = Codon optimized DNA encoding the alpha-2,6-sialyltransferaseplst6_119
  source 1..1491: mol_type = other DNA
atgaatgataatcagaataccgttgatgttgttgtgagcaccgttaacgataacgtgattgaaaataacacctaccaggtgaaaccgattgataccccgaccacctttgatagctatagttggattcagacctgtggcaccccgattctgaaagatgatgagaaatatagcctgagctttgattttgttgcaccggaactggatcaggatgaaaaattctgttttgagtttaccggtgatgtggatggtaaacgttatgttacccagaccaatctgaccgttgtggcaccgacactggaagtttatgttgatcatgcaagcctgccgagcctgcagcagctgatgaaaattatccagcagaaaaacgagtatagccagaacgaacgttttattagctggggtcgtattggtctgaccgaagataatgccgaaaaactgaatgcacatatttatccgctggcaggtaataataccagccaagaactggttgatgccgttattgattatgccgatagcaaaaatcgtctgaacctggaactgaataccaataccgcacatagctttccgaatctggcaccgattctgcgtattattagcagcaaaagcaatatcctgatcagcaacattaacctgtatgatgatggtagcgcagaatatgtgaatctgtataactggaaagacaccgaggataaaagcgttaaactgagcgatagctttctggtgctgaaagattatttcaatggcatcagcagcgaaaaaccgagcggtatttatggtcgttataattggcaccagctgtataacaccagctattactttctgcgcaaagattatctgacagttgaaccgcagctgcatgatctgcgtgaatatttaggtggtagcctgaaacaaatgagctgggatggttttagccagctgagcaaaggtgataaagaactgtttctgaacatcgtgggcttcgatcaagaaaaactgcagcaagaatatcagcagagcgaactgccgaattttgtttttaccggcaccaccacctgggcaggcggtgaaaccaaagaatattatgcacagcagcaggttaacgtggtgaataatgcaattaatgaaacgagcccgtattatctgggtcgtgaacatgacctgtttttcaaaggtcatccgcgtggtggtattatcaacgatattattctgggcagcttcaacaacatgattgacattccggcaaaagtcagctttgaagttctgatgatgaccggtatgctgccggataccgttggtggcattgcaagcagcctgtatttttcaattccggcagaaaaagtgagcttcattgtgtttaccagcagcgataccattaccgatcgtgaagatgcactgaaaagtccgctggttcaggttatgatgaccctgggtattgtgaaagaaaaagatgtgctgttttggagcgatctgccggattgtagcagcggtgtttgtattgcacagtattaa

SEQ ID NO: 18 - DNA, 1497 bp, synthetic construct
  misc_feature 1..1497: note = Codon optimized DNA encoding the alpha-2,6-sialyltransferasePpho1
  source 1..1497: mol_type = other DNA
atgagcgaagattataccccgagcatctataaactggatattaatcagaccgtgaccgatgaagaaaatgttaatctggaaccgaccaatcagagcaacatcatctttaccaaacatagctgggttcagacctgtggcacccagcaactgctgaccacacagaataaagaaagcattagcctgagcattatggcaccgcgtctggaaaaagatgagaaatattgctttgatttcaacggcgtgaataacaaaggtgatgagtataccaccaaagtgattctgaatgttgttagcccgagcctggaagtttatgttgatcatgcaagcctgccgacactgcagcagctgatggatattatcaaaagcgaagaacagaatccgaccacgcagcgttatattagctggggtcgtattcatccgaccattgagcagatgaaagaactgaatattaccagctttgtgctgggtagcaatcataccaccagtgaactggttcaggcaattgttaaacaggcacagaccaaacatcgtctgaatgtgaaactgagcagcaataccgcacatagctattataacctgattccgattctgaaagccctgaacacctttaataacgttaccgtgaccaacatcgatctgtacgatgatggtagcgcagaatatgtgaatctgtataattggcgcaacaccgagaacaaaatctataatctgcagctgggtaaagccagtctggaagatgttattagcggtgttaccgaaaactttagcggtccggcaatggcaagcatttataactggcagcaactgtatccgaccgaatatcattttctgcgcaaagattatctgaccctggaaccgtcactgcatgaactgcgtgattatctgggtgatagcctgaaacaaatgcagtgggatggctttaaaaccttcgatgtgaaacagaaagagctgttcctgagtattgtgggctttgataaacagaaactgcagaacgaatataacagcagcaatctgccgaattttgtttttaccggcaccaccgtttgggcaggtaatcatgaacgcgaatattatgcaaaacagcagatcaacgtgatcaacaacgcaattaatgaaagcagcccgtattacctgggtaaaagctatgacctgtttttcaaaggtcatcctggtggtggtattatcaataccctgattatgcagaacttcccgaagatgattgatatccctgccaaaattagctttgaggttctgatgatgaccgatatgctgccggatgcagttgcaggtatggccagcagcctgtattttaccattccgcctgataaaatcaaattcatcgttttcaccagcagcgataccattaccgatcgtgaaaccgcactgcagagtccgctggtgcaggttatgattaaactgggtattgtgaaagaagaaaacgtcctgttttgggcagatttaccgaattgcgaaaccggtatttgtattgcagcataa

SEQ ID NO: 19 - DNA, 1449 bp, synthetic construct
  misc_feature 1..1449: note = Codon optimized DNA encoding the alpha-2,6-sialyltransferaseplst6_145
  source 1..1449: mol_type = other DNA
atgaatgataatcagaataccgttgatgttgttgtgagcaccgttaacgataacgtgattgaaaataacacctaccaggtgaaaccgattgataccccgaccacctttgatagctatagttggattcagacctgtggcaccccgattctgaaagatgatgagaaatatagcctgagctttgattttgttgcaccggaactggatcaggatgaaaaattctgttttgagtttaccggtgatgtggatggtaaacgttatgttacccagaccaatctgaccgttgtggcaccgacactggaagtttatgttgatcatgcaagcctgccgagcctgcagcagctgatgaaaattatccagcagaaaaacgagtatagccagaacgaacgttttattagctggggtcgtattcgtctgaccgaagataatgccgaaaaactgaatgcacatatttatccgctggcaggtaataataccagccaagaactggttgatgccgttattgattatgccgatagcaaaaatcgtctgaacctggaactgaataccaataccggtcatagctttcgtaatattgcaccgattctgcgtgcaaccagcagcaaaaataacattctgatcagcaacattaacctgtatgatgatggtagcgcagaatatgtgagcctgtataattggaaagacaccgataacaaaagccagaaactgagcgatagctttctggtgctgaaagattatctgaatggtatcagcagcgaaaaaccgaacggtatctatagcatttataactggcatcagctgtatcacagcagctattactttctgcgtaaagattacctgaccgtggaaaccaaactgcatgatctgcgtgaatatttaggtggtagcctgaaacaaatgagctgggatacctttagccagctgtcaaaaggtgataaagaactgtttctgaacatcgtgggcttcgatcaagaaaaactgcagcaagaatatcagcagagcgaactgccgaattttgtttttaccggcaccaccacctgggcaggcggtgaaaccaaagaatattatgcacagcagcaggttaacgtggtgaataatgcaattaatgaaacgagcccgtattatctgggtcgtgaacatgacctgtttttcaaaggtcatccgcgtggtggtattatcaacgatattattctgggcagcttcaacaacatgattgacattccggcaaaagtcagctttgaagttctgatgatgaccggcatgctgccggataccgttggtggcattgcaagcagcctgtatttttcaattccggcagaaaaagtgagcttcattgtgtttaccagcagcgataccattaccgatcgtgaagatgcactgaaaagtccgctggttcaggttatgatgaccctgggtattgtgaaagaaaaagatgtgctgttttggtgctaa

SEQ ID NO: 20 - DNA, 1482 bp, synthetic construct
  misc_feature 1..1482: note = Codon optimized DNA encoding the alpha-2,6-sialyltransferase Pdam
  source 1..1482: mol_type = other DNA
atggataacaccagcctgaaagaaaccgttagcagcaatagcgcagatgttgttgaaaccgaaacctatcagctgaccccgattgatgcaccgagcagctttctgagccatagctgggaacagacctgtggcaccccgattctgaatgaaagcgataaacaggcaatcagctttgattttgttgcaccggaactgaaacaggatgagaaatattgctttaccttcaaaggcattaccggtgatcatcgttatattaccaataccacactgaccgttgtggcaccgacactggaagtttatattgatcatgcaagcctgccgagcctgcagcagctgattcatattattcaggccaaagatgaatatccgagcaatcagcgttttgttagctggaaacgtgttaccgttgatgcagataatgccaacaaactgaacattcatacctatccgctgaaaggcaataataccagtccggaaatggttgcagcaattgatgaatatgcacagagcaaaaatcgcctgaacatcgagttttatagcaataccgcacacagctttaataacctggcaagcattattcagagcctgtacaataaagataacgtgaccattagccatgtgagcctgtacgatgatggtagcgcagaatatgttaatctgtatcagtggaaagacaccccgaacaaaattgaagttctggaacgtgatattagcctgctggatgattatctggcaggcacctcaccggatacaccgaaaggtatgggtaatcgttataactggcacaaactgtatgataccgattattactttctgcgcgaagattacctggatgttgaagcaaatctgcatgatctgcgtgactatctgggtagcagcgttaaacaaatgccgtgggatgaatttgcaaaactgagcgatagccagcagaccctgtttctggatattgttggttttgataaagaacagctgcagcaacagtatagccagagtccgctgccgaattttatctttaccggcaccaccacctgggcaggcggtgaaaccaaagaatattatgcccagcagcaggttaacgtgattaataacgcaattaatgaaacgagcccgtactacctgggtaaagattatgacctgtttttcaaaggtcatcctgccggtggtgtgattaatgatattattctgggttcctttccggatatgattaacattccggcaaaaatcagtttcgaggttctgatgatgaccgatatgctgccggataccgttgcaggtattgcaagcagcctgtatttcacaattccggcagataaagtgaacttcattgtttttaccagcagcgataccattaccgatcgtgaagaagcactgaaaagtccgctggttcaggttatgctgaccctgggtattgttaaagaaaaagatgttctgttctgggcagacctgccggattgtagcagcggtgtttgtattgataaataa

SEQ ID NO: 21 - DNA, 1176 bp, synthetic construct
  misc_feature 1..1176: note = Codon optimized DNA encoding the alpha-2,6-sialyltransferasePoral_2
  source 1..1176: mol_type = other DNA
atgagcaattgggcaaaaagcgtgaccatttatctggatccggcaagcctgcctgcactgaaccagctgatggattttacccagaaatccgatgataaagaaaccgcacgtatttttggtcacacccgctttaaaatgccggaagaaattcagaaacagtaccagcatattgatatggtgggcatcaaagatgaaaaaccgaccgaagaactgtttagcattctggatcagtatccggaaagtctggaactggatctgcatatgagcattgcacatgcaaccaaactgattcagccgattctggcatatcgttttaaacatccgagccgtgttagcattcgtagcctgaatctgtatgatgatggtagcctggaatatgttggtctggaaaatctgcaggatgtggatattccgaaagcaattgcacaggcagaacagcagctggcaagctttctgatgaccggtaaagcaaaatttgataatccgattgttgcccgttatgtttggcagagccagtttccggttaaatatcattttctgagtccggaatatttcgagaaagcagcatttatcaaaccgctgaaagaatacctgcaggacaattaccagaaaatggcactgtttgcatatcaggatctgagcagcgaaaaacaggcactgtatctgaaactggtgggttttaacgatcagattaaacagctgctggaaaccaccgagaaaaagtttatctttaccggcaccaccacctgggaagcaaaaaccgatgttcgtgaatattatgcacagcagcagctgaatctgctgaaacattttacacagccgaatggcgaactgttcattggtgatgattacaaggtgtactttaaaggccatccgaaaggcgacgaaatcaatgaatatatcctgaacaacgccaaagacatcattaacattccggcaaacatcagctttgaaattctgatgatgacaggtctgctgccggataaagttggtggtattgcaagcagcctgtattttagcctgccgaaagagaaaattagccacatcattttcaccaccaacaagcaggttaaaagcaaagaagatgcactgaataacccgtacgttaaagttatgcagcgtctgggtattattgatgaaagccaggttatcttttgggacaccctgaaacagctg

SEQ ID NO: 22 - DNA, 1497 bp, synthetic construct
  misc_feature 1..1497: note = Codon optimized DNA encoding the alpha-2,6-sialyltransferase Phot
  source 1..1497: mol_type = other DNA
atgagcgaagattatacccctccgattcataaactggatattaatcagaccgtgaccgatgaagaaaacgttaaactggaaccgaccaatcagagcaacatcatctttaccaaacatagctgggttcagacctgtggcacccagcaactgctgaccacacagaataaagaaagcattagcctgagcattatggcaccgcgtctggaaaaagatgagaaatattgctttgatttcaacggcgtgaacgataaaggcgataaatacattaccaaggtgattctgaatgttgttagcccgagcctggaagtttatgttgatcatgcaagcctgcctgcactgcagcagctgatggatattatcaaaagcgaagaacagaatccgaccacgcagcgttatattagttggtggcgtattaatccgacagacgagcagatgaaagaactgaatattacccgttttccgctgatcaataatcataccagcagcgaactggttcaggcaattgttaaacaggcacagaccaaacatcgtctgaatgtgaaactgagcagcaataccgcacgtagcttttataacctgatgccgattctgaaagccctgaatacctttaataacgtgaccatcaccaacatcgatctgtacgatgatggtagcgcagaatatgtggatctgtataattggcgtaatagcgtgaacaagatctataatctgcagctgggtaaagacagtctggaagatgttattagcggtgtgaccgataactatagcggtagcgaaattgcaagcatttataactggcagcaactgtatccgaccaaatatcattttctgcgcaaagattatctgaccctggaaccgtcactgcatgaactgcgtgattatctgggtgatagcctgaaacaaatgcagtgggatggcttcaaaaaattcaacagcaaacagcaagaactgttcctgagtattgtgggctttgataaacagaaactgcagaacgaatataacagcagcaatctgccgaattttgtttttaccggcaccaccgtttgggcaggcgatcatgaaaaagaatattatgccaacaagcagatcgacgtgattgataatgcaattaatgaaagcagcccgtactatctgggtaaaagctatgacctgtttttcaaaggtcatcctggtgcgggtatcattaataccctgattatgcagaatttcccgaccatgattgatattccgagcatcattagctttgaggttctgatgatgaccgatatgctgccggatgcagttgcaggtatggcaagcagcctgtattttaccattccgagcgataaaatcaaattcatcgtgtttacctccagcgataccattaccgatcgtgaaaccgcactgcagagcgcactggtgcaggttatgattaaactgggtattgtgaaagaggaaaacgtcctgttttgggcagatttaccgaattgcgaaaccggtatttgtattgcagcataa

SEQ ID NO: 23 - DNA, 1452 bp, synthetic construct
  misc_feature 1..1452: note = Codon optimized DNA encoding the alpha-2,6-sialyltransferase Pd2
  source 1..1452: mol_type = other DNA
atgtgcaatagcgataacaccagcctgaaagaaaccgttagcagcaatagcgcagatgttgttgaaaccgaaacctatcagctgaccccgattgatgcaccgagcagctttctgagccatagctgggaacagacctgtggcaccccgattctgaatgaaagcgataaacaggcaatcagctttgattttgttgcaccggaactgaaacaggatgagaaatattgctttaccttcaaaggcattaccggtgatcatcgttatattaccaataccaccctgaccgttgttgcaccgaccctggaagtttatattgatcatgcaagcctgccgagcctgcagcagctgattcatattattcaggccaaagatgaatatccgagcaatcagcgttttgttagctggaaacgtgttaccgttgatgcagataatgccaacaaactgaacattcatacctatccgctgaaaggcaataataccagtccggaaatggttgcagcaattgatgaatatgcacagagcaaaaatcgcctgaacatcgaattctataccaataccgcacacgtgtttaataacctgcctccgattattcagccgctgtataataacgagaaagtgaaaattagccatattagcctgtatgatgatggcagcagcgaatatgttagcctgtatcagtggaaagataccccgaacaaaattgaaaccctggaaggtgaagttagcctgctggcaaattatctggcaggcaccagtccggatgcaccgaaaggtatgggtaatcgttataattggcacaaactgtatgacaccgactattactttctgcgcgaagattatctggatgttgaagcaaatctgcatgatctgcgtgattatctgggtagcagcgcaaaacaaatgccgtgggatgaatttgcaaaactgagcgatagccagcagaccctgtttctggatattgttggttttgataaagaacagctgcagcagcagtatagccagagtccgctgccgaattttatctttaccggcaccaccacctgggcaggcggtgaaaccaaagaatactatgcacagcagcaggttaacgtgattaacaatgcaattaatgaaaccagcccgtactatctgggtaaagattatgacctgtttttcaaaggtcatccggctggcggtgttattaatgatattattctgggtagcttcccggatatgattaacattccggcaaaaattagcttcgaggttctgatgatgaccgatatgctgccggataccgttgcaggtattgcaagcagcctgtatttcaccattccggcagataaagtgaactttattgttttcaccagcagcgataccattaccgatcgtgaagaagcactgaaaagtccgctggttcaggttatgctgaccctgggtattgttaaagaaaaagatgttctgttctgggcctaa

SEQ ID NO: 24 - DNA, 1488 bp, synthetic construct
  misc_feature 1..1488: note = Codon optimized DNA encoding the alpha-2,6-sialyltransferasepst6_224
  source 1..1488: mol_type = other DNA
atgaatacacagagcatcatcaaaaacgacatcaacaaaaccatcatcgacgaagaatatgtgaacctggaaccgattaatcagagcaacattagctttaccaaacatagctgggttcagacctgtggcacccagcaactgctgaccgaacagaataaagaaagcattagcctgagcgttgttgcaccgcgtctggatgatgatgagaaatattgctttgatttcaacggcgtgagcaacaaaggcgaaaaatacattaccaaagtgaccctgaatgttgtggcaccgagcctggaagtttatgttgatcatgcaagcctgccgacactgcagcagctgatggatattatcaaaagcgaagaagaaaatccgaccgcacagcgttatattgcatggggtcgtattgttccgaccgatgagcagatgaaagaactgaatattaccagctttgccctgatcaataatcatacaccggcagatctggttcaagaaattgttaaacaggcacagaccaaacatcgtctgaatgttaaactgagcagcaataccgcacatagctttgataatctggttccgattctgaaagagctgaacagctttaataacgtgaccgtgaccaatatcgatctgtacgatgatggcagcgcagaatatgttaatctgtataattggcgtgacaccctgaacaaaaccgataatctgaaaatcggcaaagactacctggaagatgtgattaatggcatcaatgaagataccagtaataccggcaccagcagcgtttataactggcagaaactgtatccggcaaactatcattttctgcgcaaagattatctgacactggaaccgtcgctgcatgaactgcgtgattatattggtgatagcctgaaacaaatgcagtgggatggcttcaaaaaattcaacagcaaacagcaagaactgttcctgagcattgtgaacttcgataaacagaaactgcagaacgaatacaatagcagcaatctgccgaattttgtgtttaccggtacaaccgtttgggcaggtaatcatgaacgcgaatattatgcaaaacagcagatcaacgtgatcaacaacgcaattaatgaaagcagtccgcattatctgggtaatagctatgacctgtttttcaaaggtcatcctggtggtggtattatcaataccctgattatgcagaattatccgagcatggttgatatcccgtccaaaattagctttgaagttctgatgatgaccgatatgctgccggatgccgttgcaggtattgcaagcagcctgtattttaccattccggcagaaaaaatcaaattcatcgttttcaccagcaccgaaaccattaccgatcgtgaaaccgcactgcgtagtccgctggttcaggttatgattaaactgggtattgtgaaagaggaaaacgtcctgttttgggcagatttaccgaattgcgaaaccggtgtttgtattgccgtttaa

SEQ ID NO: 25 - DNA, 2818 bp, synthetic construct
  misc_feature 1..2818: note = Codon optimized DNA encoding neuBCA
  source 1..2818: mol_type = other DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 26 - protein, 332 aa, Neisseria meningitidis
MQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 27 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 28 - DNA, 203 bp, synthetic construct
  misc_feature 1..203: note = Synthetic promoter sequence PmglB_70UTR_SD8
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 29 - DNA, 203 bp, synthetic construct
  misc_feature 1..203: note = Synthetic promoter sequence PmglB_70UTR_SD10
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 30 - DNA, 203 bp, synthetic construct
  misc_feature 1..203: note = Synthetic promoter sequence PmglB_54UTR
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 31 - DNA, 141 bp, synthetic construct
  misc_feature 1..141: note = Synthetic promoter sequence Plac_70UTR
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 32 - DNA, 203 bp, synthetic construct
  misc_feature 1..203: note = Synthetic promoter sequence PmglB_70UTR_SD9
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 33 - DNA, 203 bp, synthetic construct
  misc_feature 1..203: note = Synthetic promoter sequence PmglB_70UTR_SD4
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 34 - DNA, 203 bp, synthetic construct
  misc_feature 1..203: note = Synthetic promoter sequence PmglB_70UTR_SD5
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 35 - DNA, 310 bp, synthetic construct
  misc_feature 1..310: note = Synthetic promoter sequence PglpF_SD4
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 36 - DNA, 203 bp, synthetic construct
  misc_feature 1..203: note = Synthetic promoter sequence PmglB_70UTR_SD7
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 37 - DNA, 203 bp, synthetic construct
  misc_feature 1..203: note = Synthetic promoter sequence PmglB_70UTR
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 38 - DNA, 189 bp, synthetic construct
  misc_feature 1..189: note = Synthetic promoter sequence PglpA_70UTR
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 39 - DNA, 239 bp, synthetic construct
  misc_feature 1..239: note = Synthetic promoter sequence PglpT_70UTR
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 40 - DNA, 300 bp, Escherichia coli
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 41 - DNA, 310 bp, synthetic construct
  misc_feature 1..310: note = Synthetic promoter sequence PglpF_SD10
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 42 - DNA, 310 bp, synthetic construct
  misc_feature 1..310: note = Synthetic promoter sequence PglpF_SD5
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 43 - DNA, 310 bp, synthetic construct
  misc_feature 1..310: note = Synthetic promoter sequence PglpF_SD8
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 44 - DNA, 350 bp, synthetic construct
  misc_feature 1..350: note = Synthetic promoter sequence PmglB_16UTR
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 45 - DNA, 310 bp, synthetic construct
  misc_feature 1..310: note = Synthetic promoter sequence PglpF_SD9
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 46 - DNA, 300 bp, synthetic construct
  misc_feature 1..300: note = Synthetic promoter sequence PglpF_SD7
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 47 - DNA, 310 bp, synthetic construct
  misc_feature 1..310: note = Synthetic promoter sequence PglpF_SD6
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 48 - DNA, 189 bp, synthetic construct; identical to SEQ ID NO: 38
  misc_feature 1..189: note = Synthetic promoter sequence PglpA_16UTR
  source 1..189: mol_type = other DNA

SEQ ID NO: 49 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 50 - DNA, 300 bp, synthetic construct
  misc_feature 1..300: note = Synthetic promoter sequence PglpF_SD3
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 51 - DNA, 300 bp, synthetic construct
  misc_feature 1..300: note = Synthetic promoter sequence PmglpF_SD1
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

SEQ ID NO: 52 - DNA, 666 bp, synthetic construct
  source 1..666: mol_type = other DNA
atgagcctggcgatcattccggcgcgtggtggcagcaagggtatcaagaacaaaaacctggttctgctgaacaacaagccgctgatctactataccattaaagcggcgctgaacgcgaagagcatcagcaaagtggttgtgagcagcgacagcgatgagattctgaactacgcgaagagccagaacgttgacatcctgaaacgtccgattagcctggcgcaagacgataccaccagcgataaggtgctgctgcacgcgctgaagttctacaaagactatgaagatgttgtgtttctgcagccgaccagcccgctgcgtaccaacatccacattaacgaggcgttcaacctgtacaaaaacagcaacgcgaacgcgctgatcagcgttagcgaatgcgacaacaagattctgaaagcgttcgtgtgcaacgactgcggtgatctggcgggcatctgcaacgatgagtatccgtttatgccgcgtcaaaagctgccgaaaacctacatgagcaacggcgcgatctatattctgaagattaaagagttcctgaacaacccgagctttctgcagagcaagaccaaacactttctgatggacgaaagcagcagcctggacatcgattgcctggaggatctgaagaaagtggaacaaatttggaagaaataa

SEQ ID NO: 53 - DNA, 1041 bp, synthetic construct
  source 1..1041: mol_type = other DNA
atgaaagaaatcaaaatccagaacatcatcatctcggaagaaaaagccccgctggtcgtcccggaaatcggtattaaccataacggtagtctggaactggcgaaaattatggtggatgcggccttttccgcaggcgctaaaattatcaaacatcagacccacatcgtggaagacgaaatgtcaaaagcagcgaagaaagtgattccgggtaacgccaaaatctcgatctacgaaatcatgcaaaaatgcgcactggattacaaagacgaactggctctgaaagaatataccgaaaaactgggcctggtctacctgtcaacgccgttttcgcgtgcgggtgcaaaccgtctggaagatatgggtgttagtgcgttcaaaattggctccggtgaatgtaacaactatccgctgatcaaacatatcgcggcctttaaaaaaccgatgatcgttagcaccggtatgaatagcattgaatctatcaaaccgacggtcaaaattctgctggataacgaaatcccgtttgtgctgatgcataccacgaatctgtacccgaccccgcacaacctggttcgtctgaatgccatgctggaactgaagaaagaattctcatgcatggtcggcctgtcggatcacaccacggacaacctggcatgcctgggtgcagtggttctgggtgcatgtgtgctggaacgtcatttcaccgatagcatgcaccgctctggcccggatattgtttgtagcatggacacgaaagcactgaaagaactgattatccagagcgaacaaatggcgattatccgtggtaacaatgaaagcaagaaagcggctaaacaggaacaagtgaccattgactttgcattcgctagtgtcgtgtccattaaagatattaagaaaggcgaagtgctgagcatggacaatatttgggtcaaacgtccgggtctgggcggtatctctgcagcagaatttgaaaacattctgggtaagaaagcactgcgtgacattgaaaacgatgcacaactgagctatgaggattttgcgtga

SEQ ID NO: 54 - DNA, 1119 bp, synthetic construct
  source 1..1119: mol_type = other DNA
gtgaaaaagatcctgtttattacgggcagccgtgcagactactcgaaaattaaaagcctgatgtatcgtgtccagaactcgtccgaatttgaactgtatatctttgccaccggcatgcatctgagcaaaaacttcggttatacggtgaaagaactgtacaaaaacggtttcaaaaacatctacgaattcatcaactacgataaatattaccagaccgacaaagctctggcgaccacgattgatggttttagtcgttatgcaaacgaactgaaaccggatctgattgtggttcacggcgaccgtatcgaaccgctggcagcagcaattgtcggtgctctgaacaatattctggtggcgcatatcgaaggcggtgaaatttcaggcaccatcgatgactcgctgcgtcacgccattagcaaactggcacatatccacctggtcaatgatgaatttgccaaacgtcgcctgatgcaactgggcgaagacgaaaaatctatcttcatcatcggtagtccggatctggaactgctgaacgacaataaaatcagtctgtccgaagctaaaaaatactacgatatcaactacgaaaattacgcgctgctgatgtttcatccggttaccacggaaattacctcgatcaaaaaccaggctgataatctggttaaagcgctgatccaaagcaacaaaaactacatcgtcatctacccgaacaacgacctgggctttgaactgattctgcagtcttatgaagaattcaaaaacaatccgcgttttaaactgttcccgagtctgcgctttgaatacttcattaccctgctgaaaaacgctgatttcatcatcggtaatagctcttgcatcctgaaagaagccctgtatctgaaaacggcaggcattctggtgggttcccgtcagaacggccgcctgggtaacgaaaataccctgaaagttaacgccaactctgatgaaatcctgaaagcaatcaacacgatccacaaaaaacaagacctgtttagcgcaaaactggaaattctggactcaagcaaactgttcttcgaatacctgcaaagcggtgacttcttcaaactgagcacgcaaaaagtttttaaggatataaaatga

(19) United States
(12) Patent Application Publication
(10) Pub. No.: US 2026/0265709 A1
(43) Pub. Date: Sep. 10, 2026

(54) NEW SIALYLTRANSFERASES FOR IN VIVO SYNTHESIS OF LST-C

(71) Applicant: DSM IP ASSETS B.V., Maastricht (NL)

(72) Inventor: Manos PAPADAKIS, Brønshøj (DK)

(21) Appl. No.: 18/876,287

(22) PCT Filed: Jun. 20, 2023
(86) PCT No.: PCT/EP2023/066635

§ 371 Date: Dec. 18, 2024

(30) Foreign Application Priority Data
Jun. 20, 2022 (DK) PA202200591

(51) Int. Cl. C12N 9/10; C07H 1/06; C07H 5/06; C12N 15/52; C12P 19/04; C12P 19/18

(52) U.S. Cl. CPC C12N 9/1081; C07H 1/06; C07H 5/06; C12N 9/1051; C12N 15/52; C12P 19/04; C12P 19/18; C12Y 204/01222; C12Y 204/99001

(57) ABSTRACT

The present invention relates to the production of sialylated Human Milk Oligosaccharides (HMOs), in particular to the production of sialyl-lacto-N-neotetraose (LST-c), from precursor oligosaccharides and the genetic engineering of suitable cells for use in said production, as well as to methods for producing said sialylated HMOs.

### FIELD

The present invention relates to the production of sialylated Human Milk Oligosaccharides (HMOs), in particular to the production of sialyl-lacto-N-tetraose c (LST-c), and to genetically engineered cells suitable for use in said production.

### BACKGROUND

The design and construction of bacterial cell factories to produce sialylated Human Milk Oligosaccharides (HMOs), especially for more complex sialylated Human Milk Oligosaccharides (HMOs), is of paramount importance to provide innovative and scalable solutions for the more complex products of tomorrow.

To this end, rational strain engineering principles are commonly applied to single bacterial cells. Such principles usually refer to a) the introduction of a desired biosynthetic pathway to the host, b) the increase of the cellular pools of relevant activated sugars required as donors in the desired reactions, c) the enhancement of lactose import by the native lactose permease LacY and d) the introduction of suitable glycosyltransferases to facilitate the biosynthetic production of sialylated oligosaccharides (for review see Bych et al 2019, Current Opinion in Biotechnology 56:130-137).

Production of sialylated HMOs has e.g., been disclosed in WO2007/101862, describing the modifications needed to produce e.g., 3′-SL from a non-pathogenic microorganism without having to supply sialic acid to the culture resulting in a cheaper large-scale production of sialylated HMOs.

WO2019/020707 in turn describes examples of sialyltransferases expressed in a genetically modified cell, which are capable of producing complex sialylated HMOs. The sialyltransferases disclosed therein, however, only produce minor amounts of the complex sialylated HMOs, with high by-product formation.

Production of sialylated HMOs, can be hampered by side-activities of the sialyltransferases in the production strain, which may affect the ability of the cell to grow robustly even in the absence of substrate which is in turn reflected in poor yields of the sialylated HMO product.

In summary, production of sialylated HMOs, especially more complex sialylated Human Milk Oligosaccharides (HMOs), is often hampered by low production yield of the desired sialylated HMO as compared to other HMO products present after fermentation, such as HMO precursor products, as well as the simultaneous formation of other sialylated HMO species (HMO by-products), which in turn requires laborious separation procedures. Thus, sialyltransferases that are more specific towards one or more specific sialylated HMOs, in particular towards one or more specific complex sialylated HMO, are needed to lower by-product formation and to simplify product purification.

### SUMMARY

The present invention relates to a genetically modified cell comprising a recombinant nucleic acid sequence encoding an enzyme with α-2,6-sialyltransferase activity (α-2,6-sialyltransferase enzyme), capable of transferring sialic acid from an activated sugar to the terminal galactose of LNnT (acceptor) and/or to the galactose of lactose (acceptor). The genetically modified cell is capable of producing one or more HMO(s), wherein at least 10%, such as at least 11% of the total molar HMO content produced by the cell is LST-c.

In particular, the present invention relates to a genetically modified cell comprising a recombinant nucleic acid sequence encoding an enzyme with α-2,6-sialyltransferase activity selected from the group consisting of Shal (SEQ ID NO: 1), HAC1268 (SEQ ID NO: 2), Valg2 (SEQ ID NO:3) and a functional homologue of Shal (SEQ ID NO: 1), HAC1268 (SEQ ID NO: 2) or Valg2 (SEQ ID NO:3) with an amino acid sequence with at least 80% identity to an amino acid sequence selected from the group consisting of SEQ ID NO: 1, 2 and 3, respectively, and wherein said cell produces at least one sialylated Human Milk Oligosaccharide (HMO) and wherein at least 10% of the total molar HMO content produced by said cell is LST-c. I.e., at least 10%, such as at least 11% of the total molar HMO content produced by the cell is LST-c. If lactose is used as the initial substrate, the genetically modified cell may also produce 6′SL. In embodiments the level of 6′SL is below 50% of the total molar HMO produced by the cell.

The genetically modified cell according to the present invention can further comprise a promoter element that controls the expression of the recombinant nucleic acid encoding an enzyme with α-2,6-sialyltransferase activity. The sialyltransferase may e.g., be under the control of a promoter selected from the group consisting of PglpF, Plac, PmglB_70UTR PglpA_70UTR, PglpT_70UTR and variants thereof with a nucleic acid sequence selected from the group consisting of SEQ ID NOs 28 to 51, respectively. Preferably, the recombinant nucleic acid encoding an enzyme with α-2,6-sialyltransferase is under control of a strong promoter selected from the group consisting of PglpF, Plac, PmglB_70UTR, PglpA_70UTR and PglpT_70UTR with a nucleic acid sequence as shown in SEQ ID NO of 40, 49, 37, 38 or 39, respectively.

The genetically modified cell according to the present invention can further comprise a nucleic acid sequence encoding an MFS transporter protein capable of exporting the sialylated HMO into the extracellular medium.

The genetically modified cell according to the present invention can further comprise at least one recombinant nucleic acid sequence encoding at least one glycosyltransferase capable of transferring a glycosyl residue from a glycosyl donor to an acceptor oligosaccharide to produce a precursor of the sialylated human milk oligosaccharide product, such as LNnT, or to further decorate a sialylated human milk oligosaccharide to produce a more complex sialylated human milk oligosaccharide.

Further, the genetically modified cell according to the present invention typically comprises a recombinant nucleic acid sequence encoding a β-1,3-N-acetyl-glucosaminyl-transferase, such as LgtA from Neisseria meningitidis and/or a recombinant nucleic acid sequence encoding a β-1,4-galactosyltransferase, such as GalT from Helicobacter pylori.

The genetically modified cell according to the present invention can comprise a biosynthetic pathway for making a sialic acid sugar nucleotide, such as CMP-Neu5Ac. Said sialic acid sugar nucleotide pathway can be encoded by the nucleic acid sequence encoding neuBCA from Campylobacter jejuni (SEQ ID NO: 25). The nucleic acid sequence encoding neuBCA, can be encoded from a high-copy plasmid bearing the neuBCA operon. Alternatively, the neuA, neuB and neuC genes can be integrated into the genome of the genetically modified cell, preferably independently controlled by separate promoters.

Typically, the genetically modified cell according to the present invention is a microorganism, such as a bacterium or a fungus, wherein said fungus can be selected from a yeast cell, such as of the genera Komagataella, Kluyveromyces, Yarrowia, Pichia, Saccharomyces, Schizosaccharomyces or Hansenula, or from a filamentous fungous of the genera Aspergillus, Fusarium or Trichoderma, and said bacterium can be selected from the exemplified group consisting of Escherichia sp., Bacillus sp., lactobacillus sp. Corynebacterium sp. and Campylobacter sp. Accordingly, the genetically modified cell according to the present invention can be E coli.

The genetically modified cell of the present invention can be used in the production of a sialylated HMO.

Accordingly, the present invention also relates to a method for producing a sialylated human milk oligosaccharide (HMO), said method comprising culturing a genetically modified cell according to the present invention.

In addition, the invention also relates to a nucleic acid construct encoding an enzyme with α-2,6-sialyltransferase activity, such as an enzyme selected from the group consisting of Shal (SEQ ID NO: 1), HAC1268 (SEQ ID NO: 2) and Valg2 (SEQ ID NO: 3) or a functional homologue thereof with an amino acid sequence with at least 80% sequence identity to the amino acid sequence of any one of SEQ ID NO: 1, 2 or 3 respectively, wherein the enzyme encoding sequence is preferably under the control of a promoter sequence, such as a promoter selected from the group consisting of PglpF, Plac, PmglB_70UTR, PglpA_70UTR and PglpT_70UTR and variants thereof (SEQ ID NOs 28 to 51). Said nucleic acid construct is typically used in a host cell for producing a sialylated HMO, such as LST-c and/or 6′SL.

The invention additionally relates to a mixture of HMOs comprising essentially of LST-c, LNnT, 6′SL and pLNnH. Accordingly, in embodiments, the invention relates to a mixture of HMOs wherein, LST-c is in the range of 10-45 molar %, such as in the range of 10-30 molar % of the mixture, LNnT is in the range of 25-70 molar %, such as in the range of 40-70 molar % of the mixture, 6′SL is in the range of 0-65 molar %, such as in the range of 0-30 molar % of the mixture, and pLNnH is in the range of 1-20 molar %, such as in the range of 3-20 molar % of the mixture. In further embodiments, the mixture is produced according to the methods of the invention and the HMO mixture is purified such that the purified mixture contains less than 15% (w/w) lactose.

Various exemplary embodiments and details are described hereinafter, with reference to the figures and sequences when relevant. It should be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### BRIEF DESCRIPTION OF THE FIGURES

FIG. 1: Cells expressing an enzyme with an α-2,6-sialyltransferase activity that produce a molar content of LST-c (in percentage, % of total HMO) that exceeds LST-c levels produced by cells expressing Pdam (SEQ ID NO: 8), which has been suggested in the prior art to be able to sialylate LNnT.

FIG. 2: Overview of the synthesis of LST-c mixtures.

### DETAILED DESCRIPTION

The present invention approaches the biotechnological challenges of in vivo HMO production, in particular, of sialylated HMOs which contain at least one sialyl monosaccharide, such as the sialylated HMOs LST-c and 6′SL. The present invention offers specific strain engineering solutions to produce specific complex sialylated HMOs, in particular LST-c, by exploiting the substrate specificity towards the terminal galactose moiety on LNnT and the activity of the α-2,6-sialyltransferases of the present disclosure.

A genetically modified cell of the present invention expresses genes encoding key enzymes for sialylated HMO biosynthesis, in some embodiments along with one or more genes encoding a biosynthetic pathway for making a sialic acid sugar nucleotide, such as the neuBCA operon from Campylobacter jejuni shown in SEQ ID NO: 25, allowing for formation of CMP-N-acetylneuraminic acid, which enables the cell to produce a sialylated oligosaccharide from one or more oligosaccharide substrates, such as lactose, LNT-II and/or LNnT. Depending on the substrate, one or more additional glycosyltransferases and pathways for making nucleotide-activated sugars, such as glucose-UDP-GlcNac, GDP-fucose, UDP-galactose, UDP-glucose, UDP-N-acetylglucosamine, UDP-N-acetylgalactosamine and CMP-N-acetylneuraminic acid can also be present in the genetically modified cell.

In particular, the sialylated HMO(s) produced is LST-c and/or 6′SL.

The advantage of using any one of the α-2,6-sialyltransferases of the present disclosure in the present context is their ability to recognize and sialylate, not only lactose to generate 6′SL, but also larger oligosaccharides, such as LNnT, to generate LST-c. In particular, the present disclosure describes enzymes with α-2,6-sialyltransferase activity (α-2,6-sialyltransferases) that are more active on the terminal galactose of LNnT than α-2,6-sialyltransferases described in the prior art, such as Plst6_119 (see WO2019/020707) and Pdam (see WO2021/1123113).

The traits of the α-2,6-sialyltransferases described herein are therefore well-suited for high-level industrial production of LST-c either as the predominant sialylated HMO or for the simultaneous formation of other sialylated HMOs, such as 6′SL and other by-product HMOs, to produce desired mixtures of sialylated oligosaccharide products.

The genetically modified cells of the present invention, which express an α-2,6-sialyltransferase with high specificity towards the terminal galactose of LNnT, for the first time enable the production of high titers of LST-c. Thereby, the present invention enables a more efficient LST-c production, which is highly beneficial in biotechnological production of more complex sialylated HMOs, such as LST-c. In particular some of the enzymes described herein are suitable for producing LST-c with low 6′SL by-product formation and other enzymes are suitable of producing mixtures of 6′SL and LST-c, which may also be beneficial for products where both HMOs are desired.

In the following sections, individual elements of the invention, and in particular of the genetically modified cell is described, it is understood that these elements can be combined across the individual sections.

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry 2015 vol 72.

The present invention focuses on sialylated HMO's, which are generally acidic. Examples of acidic HMOs include 3′-sialyllactose (3′SL), 6′-sialyllactose (6′SL), 3-fucosyl-3′-sialyllactose (FSL), 3′-O-sialyllacto-N-tetraose a (LST-a), fucosyl-LST-a (FLST-a), 6′-sialyllacto-N-tetraose b (LST-b), fucosyl-LST b (FLST b), 6′-sialyllacto-N-neotetraose (LST-c), fucosyl-LST-c (FLST-c), 3′-sialyllacto-N-neotetraose (LST-d), fucosyl-LST d (FLST-d), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT).

In the context of the present invention, complex HMOs are composed of at least 4 monosaccharide units, preferably at least 5 monosaccharide units. Preferably, in one embodiment, a complex HMO is one that require at least two different glycosyltransferase activities to be produced from lactose as the initial substrate, e.g., the formation of LST-c requires an alpha-2,6-sialyltransferase, a β-1,3-N-acetyl-glucosaminyl-transferase and a β-1,4-galactosyltransferase.

In one aspect according to the present invention, the human milk oligosaccharide (HMO) is an acidic HMO such as a sialylated HMO. The sialylated HMO in one aspect comprises at least three monosaccharide units, such as three, four, five or six monosaccharide units.

In one aspect of the present invention, the sialylated human milk oligosaccharide (HMO) produced by the cell is a sialylated HMO selected from the list consisting of 6′SL, and LST-c. In a further aspect of the present invention, the sialylated human milk oligosaccharide (HMO) produced by the cell is an HMO of at least five monosaccharide units, such as LST-c.

In one embodiment the one or more HMO produced by the method and/or genetically engineered cell disclosed herein is a mixture of one or more of the following HMOs LNT-II LNnT, pLNnH, 6′SL and LST-c.

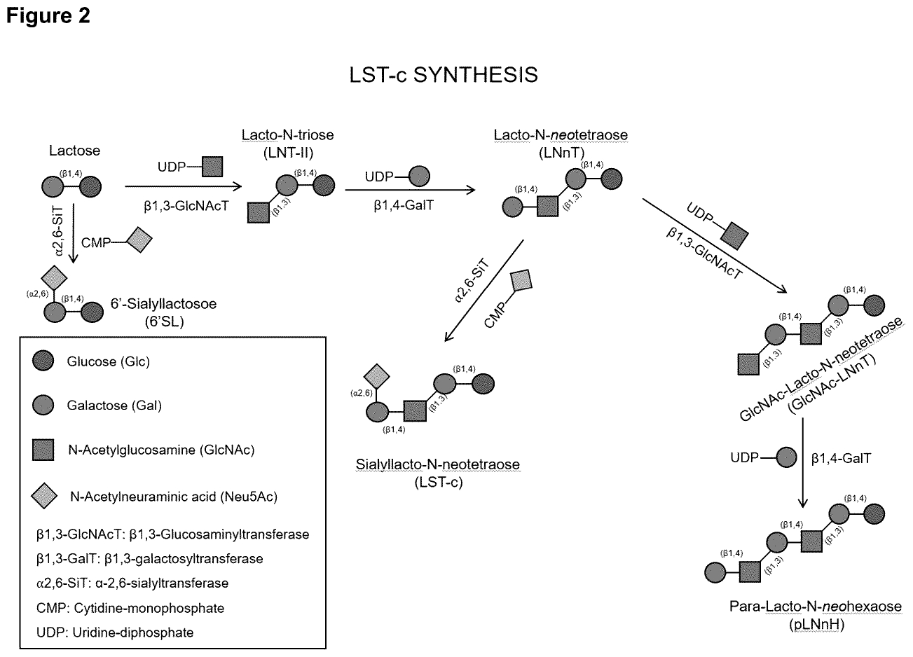

In one embodiment LST-c is the desired product produced by the method and/or genetically engineered cell disclosed herein, and the other oligosaccharides (such as HMOs) produced by the process depicted in FIG. 2 are considered by-product oligosaccharides or by-product HMOs. Examples of by-product HMOs in the context of producing high concentrations of LST-c is 6′SL, LNT-II, LNnT and pLNnH. The product HMO, in this case LST-c, may be purified to reduce or eliminate the by-product HMOs from the final product.

In another embodiment a mixture of sialylated HMOs, such as a mixture of LST-c and 6′SL, is the desired product produced by the method and/or genetically engineered cell disclosed herein, and the other oligosaccharides (such as HMOs) produced by the process depicted in FIG. 2 are considered by-product oligosaccharides or by-product HMOs. Examples of by-product HMOs in the context of producing mixtures of sialylated HMOs is LNT-II, LNnT and pLNnH. The product HMOs, in this case is LST-c and 6′SL, which may be purified to reduce or eliminate the by-product HMOs from the final product

Production of these HMO's may require the presence of two or more glycosyltransferase activities, in particular if starting from lactose as the acceptor oligosaccharide.

### An Acceptor Oligosaccharide

A genetically modified cell according to the present invention comprises a recombinant nucleic acid sequence encoding an enzyme with α-2,6-sialyltransferase activity capable of transferring sialic acid from an activated sugar to the terminal galactose of an acceptor oligosaccharide.

In the context of the present invention, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making a more complex HMO and can also be termed the precursor molecule.

The acceptor oligosaccharide can be either an intermediate product of the present fermentation process, an end-product of a separate fermentation process employing a separate genetically modified cell, or an enzymatically or chemically produced molecule.

In the present context, said acceptor oligosaccharide for the α-2,6-sialyltransferase is preferably lacto-N-neotetraose (LNnT), which is produced from the precursor molecules lactose (e.g., acceptor for the β-1,3-N-acetyl-glucosaminyl-transferase) and/or lacto-N-triose II (LNT-II) (e.g., acceptor for the β-1,4-galactosyltransferase). The precursor molecule is preferably fed to the genetically modified cell which is capable of producing LNnT from the precursor.

### Glycosyltransferases

The genetically modified cell according to the present invention comprises at least one recombinant nucleic acid sequence encoding at least one glycosyltransferase capable of transferring a sialyl residue from a sialyl donor to an acceptor oligosaccharide to synthesize a sialylated human milk oligosaccharide product, i.e., a sialyltransferase.

The genetically modified cell according to the present invention may comprise at least one further recombinant nucleic acid sequence encoding at least one glycosyltransferase capable of transferring a glycosyl residue from a glycosyl donor to an acceptor oligosaccharide. Preferably, the additional glycosyltransferase(s) enables the genetically modified cell to synthesize LNnT from a precursor molecule, such as lactose or LNT-II.

The additional glycosyltransferase is preferably selected from the group consisting of, galactosyltransferases, glucosaminyltransferases, sialyltransferases, N-acetylglucosaminyl transferases and N-acetylglucosaminyl transferases.

In one aspect, the sialyltransferase in the genetically modified cell of the present invention is an α-2,6-sialyltransferase. Preferably, the α-2,6-sialyltransferase is capable of transferring a sialic acid unit onto the terminal galactose of an LNnT molecule.

In the present invention, the at least one functional enzyme (α-2,6-sialyltransferase) capable of transferring a sialyl moiety from a sialyl donor to an acceptor oligosaccharide can be selected from the list consisting of Shal, HAC1268, Valg2, Pmult and Plst6_119 (table 1). These enzymes can e.g., be used to produce 6′SL and/or LST-c, respectively.

In preferred embodiments the α-2,6-sialyltransferase is selected from the group consisting of Shal, HAC1268, and Valg2.

Without being bound by theory, an α-2,6-sialyltransferase with a higher affinity for the terminal galactose moiety in LNnT compared to the affinity for the terminal galactose moiety in lactose can be advantageous as such an α-2,6-sialyltransferase would in theory produce less 6′SL when the initial substrate is lactose and wherein the availability of LNnT is in such a case is not limited. A lower amount of 6′SL would in such a case result in a more beneficial purification of LST-c, as the purification of LST-c from a mixture of HMOs comprising mostly neutral HMO side products and LST-c would be simpler, as it is easier to separate neutral HMO(s) from acidic HMO(s) than separating different acidic HMOs. Hence a lower initial amount of 6′SL is considered benefit in the purification of LST-c.

In preferred embodiments, the α-2,6-sialyltransferase of the present invention results in an LST-c formation that exceeds the formation of 6′SL, when lactose is the initial substrate. In a further preferred embodiment, the α-2,6-sialyltransferase is HAC1268.

In embodiments, the expression of an α-2,6-sialyltransferase of the invention in a genetically modified cell is further combined with expression of an a β-1,4-galactosyltransferase, such as galT from Helicobacter pylori. In a further embodiment, a third enzyme is added, such as a β-1,3-N-acetyl-glucosaminyl-transferase, e.g., LgtA from Neisseria meningitidis.

Exemplified glycosyltransferases are preferably selected from the glycosyltransferases described below.

### α-2,6-Sialyltransferase

An alpha-2,6-sialyltransferase refers to a glycosyltransferase that catalyzes the transfer of sialyl from a donor substrate, such as CMP-N-acetylneuraminic acid, to an acceptor molecule in an alpha-2,6-linkage. Preferably, an alpha-2,6-sialyltransferase used herein does not originate in the species of the genetically engineered cell, i.e., the gene encoding the alpha-2,6-sialyltransferase is of heterologous origin and is selected from an alpha-2,6-sialyltransferase identified in table 1. In the context of the present invention, the acceptor molecule for the alpha-2,6-sialyltransferase is lactose and/or an acceptor oligosaccharide of at least four monosaccharide units, e.g., LNnT. Heterologous alpha 2,6-sialyltransferases that are capable of transferring a sialyl moiety onto LNnT are known in the art, two of which are identified in table 1.

The α-2,6-sialyltransferases investigated in the present application are listed in table 3. Of the α-2,6-sialyltransferases investigated (table 3), only the α-2,6-sialyltransferases listed in table 1 were capable of producing LST-c. The sialyltransferase can be selected from an amino acid sequence with at least 80%, such as at least 85%, such as at least 90%, such as at least 95%, or such as at least 99% sequence identity to the amino acid sequence of any one of the alpha-2,6-sialyltransferases listed in table 1.

TABLE 1
List of alpha-2,6-sialyltransferase enzymes capable of producing LST-c

| Enzyme | | SEQ | | |
|---|---|---|---|---|
| Name | GenBank ID¹ | ID NO: | Origin | Ref |
| Shal | WP_108944675.1 | 1 | Shewanella halifaxensis | |
| HAC1268 | STP03859.1 | 2 | Helicobacter acinonychis str. Sheeba | |
| valg2 | WP_169629213.1 | 3 | Vibrio alginolyticus | |
| pmult | WP_005753497.1 | 4 | Pasteurela multocida | |
| plst6_119 | BAI49484.1 | 5 | Photobacterium leiognathi JT-SHIZ-119 | WO 2019/020707² |
| ppho1 | WP_065208987.1 | 6 | Photobacterium phosphoreum | |
| plst6_145 | BAF91416.1 | 7 | Photobacterium leiognathi JT-SHIZ-145 | |
| pdam | WP_151182185.1 | 8 | Photobacterium damselae | WO2021/123113³ |
| Poral_2 | WP_101774701.1 | 9 | Pasteurella oralis | |
| phot | WP_036792497.1 | 10 | Photobacterium kishitanii | |
| Pd2 | BAA25316.1 | 11 | Photobacterium damsela JT0160 | WO2021/123113³ |
| pst6-224 | BAF92026.1 | 12 | Photobacterium sp JT-ISH-224 | |

¹The GenBank ID's reflect the full length enzymes, in the present invention truncated or mutated versions may have been used, these are represented by the sequences indicated by the SEQ ID NOs.
²SEQ ID NO: 28 of WO 2019/020707 is 99.6% identical to SEQ ID NO: 5. There is no evidence suggesting that this enzyme can produce LST-c.
³SEQ ID NO: 32 of WO2021/123113 is truncated with 93 amino acids in the N-terminal and 208 amino acids in the c-terminal leading to 59.1% identity with SEQ ID NO: 11, the overlapping region is however 100% identical. Furthermore, SEQ ID NO: 32 of WO2021/123113 is truncated with 90 amino acids in the N-terminal and 13 amino acids in the c-terminal leading to 75.1% identity with SEQ ID NO: 8. The overlapping region is 94.9% identical to SEQ ID NO 8.

Example 1 of the present invention has identified the heterologous alpha-2,6-sialyltransferases Shal, HAC1268 and Valg2 (SEQ ID NO: 1, 2 and 3, respectively), which are capable of producing higher LST-c titers when introduced into an LNnT producing cell, than the previously known alpha-2,6-sialyltransferases Plst6_116 and Pdam.

Furthermore, the experiments performed in Example 1 identified the heterologous alpha-2,6-sialyltransferases Shal, HAC1268, Valg2, pmult, plst6_119 (SEQ ID NO: 1, 2, 3, 4 and 5, respectively), as being capable of producing higher LST-c titers when introduced into an LNnT producing cell, than the previously known alpha-2,6-sialyltransferase Pdam. In addition, even though plst6_119 is known to produce 6′SL (WO 2019/020707) it has to our knowledge not previously been used in a method for producing LST-c.

In the examples Shal, HAC1268 Valg2, pmult and plst6_119 are used in combination with LgtA from Neisseria meningitidis and galT from Helicobacter pylori to produce a mixture of LST-c and 6′SL starting from lactose as substrate. Shal, HAC1268, Valg2, pmult and plst6_119 may alternatively be combined with galT from Helicobacter pylori to produce LST-c starting from LNT-II as substrate, this could eliminate the formation of 6′SL. Additionally, Shal, HAC1268 Valg2, pmult and plst6_119 may be sufficient to produce LST-c when starting from LNnT.

If desired, the alpha-2,6-sialyltransferases identified in table 1, may also be used in a modified strain without β-1,3-N-acetyl-glucosaminyl-transferase and β-1,3-galactosyltransferase activity, resulting in the production of 6′SL without the presence of LST-c when using lactose as substrate.

In embodiments the genetically modified may comprise more than one copy, such as two copies or three copies of the nucleic acid sequence encoding the α-2,6-sialyltransferase enzyme. In a preferred embodiment the nucleic acid sequence (s) encoding the α-2,6-sialyltransferase is integrated into the genome of the modified cell.

In one embodiment of the invention, the enzyme with α-2,6-sialyltransferase activity is Shal from Shewanella halifaxensis comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1. In embodiments the Shal enzyme is able to produce 6′SL:LST-c ratio in a ratio between 1:1 and 3:1 when introduced into a cell capable of producing LNnT from lactose. In embodiments the Shal enzyme is able to produce at least 15%, such as at least 20% of LST-c of the total HMO and at least 20%, such as at least 30%, such as at least 40%, such as at least 50%, such as at least 60% of 6′SL of the total HMO.

In another embodiment of the invention, the enzyme with α-2,6-sialyltransferase activity is HAC1268 from Helicobacter acinonychis str. Sheeba comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2. The genetically modified cell according to any of the preceding embodiments, wherein the α-2,6-sialyltransferase enzyme is HAC1268 and the LST-c:6′SL ratio is above 2:1, preferably above 3.5:1, preferably above 15:1 when introduced into a cell capable of producing LNnT from lactose. In embodiments the HAC1268 enzyme is able to produce at least 15%, such as at least 20%, such as at least 30%, such as at least 40% of LST-c of the total HMO and less than 10%, such as less than 8%, such as less than 5% of 6′SL of the total HMO.

In another embodiment of the invention, the enzyme with α-2,6-sialyltransferase activity is Valg2 from Vibrio alginolyticus comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3. In embodiments the Valg2 enzyme is able to produce 6′SL:LST-c ratio in a ratio between 1:1 and 3:1 when introduced into a cell capable of producing LNnT from lactose. In embodiments the Valg2 enzyme is able to produce at least 10%, such as at least 15%, such as at least 20% of LST-c of the total HMO and at least 10%, such as at least 15%, such as at least 20%, such as at least 25% of 6′SL of the total HMO.

In another embodiment of the invention, the enzyme with α-2,6-sialyltransferase activity is pmult from Pasteurella multocida comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4.

In another embodiment of the invention, the enzyme with α-2,6-sialyltransferase activity is plst6_119 from Photobacterium leiognathid comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5. The plst6_119 is in particular introduced into a genetically modified cell which further comprises a β-1,4-galactosyltransferase and preferably also a β-1,3-N-acetyl-glucosaminyl-transferase.

### β-1,3-N-Acetyl-Glucosaminyl-Transferase

A β-1,3-N-acetyl-glucosaminyl-transferase is any protein which comprises the ability of transferring the N-acetyl-glucosamine of UDP-N-acetyl-glucosamine to lactose or another acceptor molecule, in a beta-1,3-linkage. Preferably the β-1,3-N-acetyl-glucosaminyl-transferase used herein does not originate in the species of the genetically engineered cell, i.e., the gene encoding the β-1,3-galactosyltransferase is of heterologous origin. In the context of the present invention, the acceptor molecule is either lactose or an oligosaccharide of at least four monosaccharide units, e.g., LNnT, or more complex HMO structures.

Non-limiting examples of β-1,3-N-acetyl-glucosaminyltransferases are given in table 5. β-1,3-N-acetyl-glucosaminyltransferase variants may also be useful, preferably such variants are at least 80%, such as at least 85%, such as at least 90, such as at least 95% identical to the amino acid sequence of any one of the β-1,3-N-acetyl-glucosaminyltransferase in table 5.

TABLE 5
List of β-1,3-N-acetyl-glucosaminyltransferase

| Protein Name | GenBank ID | origin |
|---|---|---|
| IgtA | WP_033911473.1 | Neisseria meningitidis |
| | WP_002248149 | |
| | AAF42258.1 | |
| | ELK60643.1 | |
| IgtA | AAK70338.1 | Neisseria gonorrhoeae |
| | ACF31229.1 | |
| IgtA | AAK02595.1 | Pasteurella multocida |
| IgtA | EEZ72046.1 | Neisseria cinerea |
| HD0466 | WP_010944479.1 | Haemophilus ducreyi |
| PmnagT | WP_014390683.1 | Pasteurella multocida |

In one embodiment, the recombinant nucleic acid sequence encoding a β-1,3-N-acetylglucosaminyltransferase comprises or consists of the amino acid sequence of SEQ ID NO: 26 (LgtA from N. meningitidis) or a functional homologue thereof with an amino acid sequence with at least 80%, such as at least 85%, such as at least 90%, such as at least 95%, or such as at least 99% sequence identity to SEQ ID NO: 26.

For the production of LNnT from lactose as substrate, the LNT-II precursor is formed using a β-1,3-N-acetylglucosaminyltransferase. In one embodiment the genetically modified cell comprises a β-1,3-N-acetylglucosaminyltransferase gene, or a functional homologue or fragment thereof, to produce the intermediate LNT-II from lactose.

Some of the examples below use the heterologous β-1,3-N-acetyl-glucosaminyl-transferase named LgtA from Neisseria meningitidis or a variant thereof.

### β-1,4-Galactosyltransferase

A β-1,4-galactosyltransferase is any protein that comprises the ability of transferring the galactose of UDP-Galactose to a N-acetyl-glucosaminyl moiety to an acceptor molecule in a beta-1,4-linkage. Preferably, a β-1,4-galactosyltransferase used herein does not originate in the species of the genetically engineered cell i.e., the gene encoding the β-1,4-galactosyltransferase is of heterologous origin. In the context of the present invention the acceptor molecule, is an acceptor saccharide, e.g., LNT-II, or more complex HMO structures.

The examples below use the heterologous β-1,4-galactosyltransferase named GalT or a variant thereof, to produce e.g., LST-c in combination with other glycosyl transferases.

Non-limiting examples of β-1,4-galactosyltransferases are provided in table 6. β-1,4-galactosyltransferases variants may also be useful, preferably such variants are at least 80%, such as at least 85%, such as at least 90, such as at least 95% identical to the amino acid sequence of any one of the β-1,4-galactosyltransferases in table 6.

TABLE 6
List of beta-1,4-glycosyltransferases

| Protein Name | GenBank ID | Origin |
|---|---|---|
| GalT | WP_001262061.1 | Helicobacter pylori |
| LgtB | AAF42257.1 | Neisseria meningitidis MC58 |

In one embodiment, the recombinant nucleic acid sequence encoding a β-1,4-galactosyltransferases comprises or consists of the amino acid sequence of SEQ ID NO: 27 (galT from H. pylori) or a functional homologue thereof with an amino acid sequence with at least 80%, such as at least 85%, such as at least 90%, such as at least 95%, or such as at least 99% sequence identity to SEQ ID NO: 27.

To produce LNnT form an LNT-II precursor, a β-1,4-galactosyltransferase is needed. In one embodiment, the genetically modified cell comprises a β-1,4-galactosyltransferase gene, or a functional homologue or fragment thereof.

Below are non-limiting examples of genetically modified strains according to the present invention with specific combinations of glycosyl transferases that will lead to production of LST-c using lactose as initial substrate.

In a non-limiting example, LgtA from Neisseria meningitidis is used in combination with galT from Helicobacter pylori and Shal from Shewanella halifaxensis to produce LST-c starting from lactose as initial substrate.

In a non-limiting example, LgtA from Neisseria meningitidis is used in combination with galT from Helicobacter pylori and HAC1268 from Helicobacter acinonychis str. Sheeba to produce LST-c starting from lactose as initial substrate.

In a non-limiting example, LgtA from Neisseria meningitidis is used in combination with galT from Helicobacter pylori and Valg2 from Vibrio alginolyticus to produce LST-c starting from lactose as initial substrate.

In a non-limiting example, LgtA from Neisseria meningitidis is used in combination with galT from Helicobacter pylori and pmult from Pasteurella multocida to produce LST-c starting from lactose as initial substrate.

In a non-limiting example, LgtA from Neisseria meningitidis is used in combination with galT from Helicobacter pylori and plst6_119 from Photobacterium leiognathid to produce LST-c starting from lactose as initial substrate.

In a non-limiting example, galT from Helicobacter pylori is used in combination with Shal from Shewanella halifaxensis to produce LST-c starting from LNT-II as initial substrate.

In a non-limiting example, galT from Helicobacter pylori is used in combination with HAC1268 from Helicobacter acinonychis str. Sheeba to produce LST-c starting from LNT-II as initial substrate.

In a non-limiting example, galT from Helicobacter pylori is used in combination with Valg2 from Vibrio alginolyticus to produce LST-c starting from LNT-II as initial substrate.

In a non-limiting example, galT from Helicobacter pylori is used in combination with pmult from Pasteurella multocida to produce LST-c starting from LNT-II as initial substrate. In a non-limiting example, galT from Helicobacter pylori is used in combination with plst6_119 from Photobacterium leiognathid to produce LST-c starting from LNT-II as initial substrate.

### Glycosyl-Donor—Nucleotide-Activated Sugar Pathways

When carrying out the method of this invention, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably the following activated sugar nucleotides are involved in the glycosyl transfer: glucose-UDP-GlcNAc, UDP-galactose, UDP-glucose, UDP-N-acetylglucosamine, UDP-N-acetylgalactosamine (GlcNAc) and CMP-N-acetylneuraminic acid.

The genetically modified cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the method, the genetically modified cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Sialic Acid Sugar Nucleotide Synthesis Pathway

Preferably, the genetically modified cell according to the present invention comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the alpha-2,6-sialyltransferase of the present invention. E.g., the genetically modified cell comprises a sialic acid synthetic capability through provision of an exogenous UDP-GlcNAc 2-epimerase (e.g., neuC of Campylobacter jejuni (GenBank AAK91727.1) or equivalent (e.g., (GenBank CAR04561.1), a Neu5Ac synthase (e.g., neuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., neuA of C. jejuni (GenBank AAK91728.1) or equivalent, (e.g., Vibrio brasiliensis CMP-sialic acid synthase, GenBank WP_006881452.1).

In one or more examples UDP-GlcNAc 2-epimerase, CMP-Neu5Ac synthetase, Neu5Ac synthase from Campylobacter jejuni, also referred to as neuBCA from Campylobacter jejuni or simply the neuBCA operon, may be plasmid borne or integrated into the genome of the genetically modified cell. In some embodiments, the sialic acid sugar nucleotide pathway is encoded by the nucleic acid sequence encoding neuBCA from Campylobacter jejuni (SEQ ID NO: 25) or a functional variant thereof having nucleic acid sequence which is at least 80% identical, such as at least 85%, such as at least 90% or such as at least 99% to SEQ ID NO: 25.

Additionally, the nucleic acid sequence encoding neuBCA is preferably encoded from a high-copy plasmid bearing the neuBCA operon. In embodiments, the high-copy plasmid is the BlueScribe M13 plasmid (pBS). In relation to the present invention, a high-copy plasmid is a plasmid that that replicates to a copy number above 50 when introduced into the cell.

In other embodiments the pathway for making a sialic acid sugar nucleotide (CMP-Neu5Ac) is encoded from genomically integrated neuA, neuB and neuC nucleic acid sequences, wherein the three genes preferably are under control of individual promoters. The neuA, neuB and neuC genes introduced into the genome of the genetically engineered cell may for example be codon optimized neuC of Campylobacter jejuni (GenBank AAK91727.1), codon optimized neuB of C. jejuni (GenBank AAK91726.1) and codon optimized neuA of C. jejuni (GenBank AAK91728.1) or equivalents thereof as indicated above. In one embodiment the codon optimized neuA is SEQ ID NO: 52, neuB is SEQ ID NO: 53) and neuC is SEQ ID NO: 54 or functional variants thereof. Preferably, the integrated genes are independently under control of a promoter selected from table 2, more preferably a strong promoter selected from the group consisting of SEQ ID NOs 28, 29, 30, 31, 32, 33, 34, 35, 36, 37, 38, 39, 40, 41, 42, 43 and 44. In one embodiment the neuA, neuB and neuC genes are independently under control of the PglpF promoter (SEQ ID NO: 40), and the genes are preferably heterologous.

In embodiments the genetically modified cell comprises both a genomically integrated pathway for making a sialic acid sugar nucleotide (CMP-Neu5Ac), as well as a plasmid bearing the neuBCA operon for increased production of CMP-Neu5Ac.

### A Deficient Sialic Acid Catabolic Pathway

The genetically modified cell of the present invention preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1 (GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanK are mutated, while nanE remains functional. In another preferred embodiment, nanA and nanE are mutated, while nanK has not been mutated, inactivated or deleted. A mutation is one or more changes in the nucleic acid sequence coding the gene product of nanA, nanK, nanE, and/or nanT. E.g., the mutation may be 1, 2, up to 5, up to 10, up to 25, up to 50 or up to 100 changes in the nucleic acid sequence. E.g., the nanA, nanK, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present invention, nanA, nanK, nanE, and/or nanT genes are preferably inactivated.

### Major Facilitator Superfamily (MFS) Transporter Proteins

The oligosaccharide product, such as the HMO produced by the cell, can be accumulated both in the intra- and the extracellular matrix. The product can be transported to the supernatant in a passive way, i.e., it diffuses outside across the cell membrane. The more complex HMO products may remain in the cell, which is likely to eventually impair cellular growth, thereby affecting the possible total yield of the product from a single fermentation. The HMO transport can be facilitated by major facilitator superfamily transporter proteins that promote the effluence of sugar derivatives from the cell to the supernatant. The major facilitator superfamily transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically modified cell according to the present invention can further comprise a nucleic acid sequence encoding a major facilitator superfamily transporter protein capable of exporting the sialylated human milk oligosaccharide product or products.

In the resent years, several new and efficient major facilitator superfamily transporter proteins have been identified, each having specificity for different recombinantly produced HMOs and development of recombinant cells expressing said proteins are advantageous for high scale industrial HMO manufacturing. WO2021/123113 claim different E. coli and heterologous transporters for the export of 3′SL, 6′SL and LST-c.

Thus, in one or more exemplary embodiments, the genetically engineered cell according to the method described herein further comprises a gene product that acts as a major facilitator superfamily transporter. The gene product that acts as a major facilitator superfamily transporter may be encoded by a recombinant nucleic acid sequence that is expressed in the genetically engineered cell. The recombinant nucleic acid sequence encoding a major facilitator superfamily transporter, may be integrated into the genome of the genetically engineered cell, or expressed using a plasmid.

In one embodiment, the genetically modified cell of the invention comprises a nucleic acid sequence encoding a major facilitator superfamily transporter protein capable of exporting the sialylated human milk oligosaccharide product into the extracellular medium, in particular, the transporters with specificity towards LST-c and/or 6′SL are preferred.

### HMO Concentrations

The genetically modified cell comprising more than one glycosyltransferase described herein will generally produce a mixture of HMOs as a result of the multistep process towards the final HMO product. In the production of LST-c from lactose as the initial substrate, it is expected that 6′SL (sialylated lactose), LNT-II, LNnT, LST-c and pLNnH are present at the end of the cultivation.

The HMO products produced by the methods disclosed herein can be described by their ratios in a mixture of HMOs. The “ratio” as described herein is understood as the ratio between two amounts of HMOs, such as, but not limited to, the amount of one HMO divided by the amount of the other HMO, or the amount of one HMO divided by the total amount of HMOs.

In one embodiment described herein, following cultivation of the genetically modified cell as described herein, the mixture of HMOs has a molar % of LST-c between 10% to 45%, such as between 10% to 30% and 6′SL between 0 and 65% such as between 4% to 50%, such as molar % of LST-c between 11% to 25% and 6′SL between 5% to 30%. In a preferred embodiment, the molar % of LST-c is above 11%, such as above 15%, such as above 18%, such as above 25%, such as above 35%, such as above 40% of the total HMO. In a further preferred embodiment, the molar % of 6′SL is below 65%, such as below 50%, such as below 40%, such as below 30%, such as below 20%, such as below 10%, such as below 5% of the total HMO.

In some embodiments described herein, following cultivation of the genetically modified cell a mixture of 6′SL and LST-c is produces, preferably the cell produces a ratio of 6′SL: LST-c between 1:1 and 4:1, such as from 1.5:1 to 3:1.

In some embodiments described herein, following cultivation of the genetically modified cell LST-c is produced with low amounts of 6′SL by-product, preferably the cell produces a ratio of LST-c:6′SL above 2:1, such as a above 3.5:1, preferably above 15:1, such as between 5:1 to 20:1. In an even more preferred embodiment the cell does not produce any 6′SL at all.

In some embodiments, the genetically modified cell of the present invention expresses Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, and the molar % content of LST-c produced by the genetically modified cell is above 11%, such as above 15%, such as above 25% of the total HMO.

In some embodiments, the genetically modified cell of the present invention expresses HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the molar % content of LST-c produced by the genetically modified cell is above 11%, such as above 15%, such as above 20%, such as above 35%, such as above 40% of the total HMO.

In some embodiments, the genetically modified cell of the present invention expresses Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3, and the molar % content of LST-c produced by the genetically modified cell is above 11% of the total HMO.

In some embodiments, the genetically modified cell of the present invention expresses Pmult comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4, and the molar % content of LST-c produced by the genetically modified cell is above 10% of the total HMO.

In some embodiments, the genetically modified cell of the present invention expresses Plst6_119 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5, and the molar % content of LST-c produced by the genetically modified cell is above 10% of the total HMO.

The molar % ratios supported by experimental data from the Examples shows exemplary HMO composition ranges, wherein the ratio of LST-c:6′SL is in the range from 1:5 to 4:1.

In a some embodiments the ratio of LST-c:6′SL does not constitute more than 2-fold 6′SL over LST-c, so the LST-c:6′SL ratio is not lower than 1:2, preferably not lower than 1:1.5. More preferably the LST-c:6′SL ratio is above 1:1, such as above 2:1, such as above 3:1.

In some embodiments, the genetically modified cell of the present invention expresses HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the ratio of LST-c:6′SL is above 3:1, i.e. the genetically modified cell produce more than 15% LST-c and/or less than 5% 6′SL of the total HMO, such as more than 30% LST-c and/or less than 10% 6′SL of the total HMO.

In some embodiments, the genetically modified cell of the present invention expresses HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and the molar % content of 6′SL produced by the genetically modified cell is below 15%, such as below 10%, such as below 5% of the total HMO.

### The Genetically Modified Cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering and inclusion of MFS transporters as described in the above sections, which the skilled person will know how to combine into a genetically modified cell capable of producing one or more sialylated HMO's.

In one aspect of the invention, the genetically modified cell comprises a recombinant nucleic acid sequence encoding an enzyme with α-2,6-sialyltransferase activity, which is capable of producing at least 10% LST-c of the total molar HMO content produced by the cell.

In one embodiment the genetically modified cell capable of producing a sialylated HMO, comprises a recombinant nucleic acid sequence encoding an enzyme with α-2,6-sialyltransferase activity, wherein said enzyme is selected from the group consisting of:
a. Shal comprising or consisting of the amino acid sequence of SEQ ID NO: 1 or an amino acid sequence with at least 80% identity to SEQ ID NO: 1,
b. HAC1268 comprising or consisting of the amino acid sequence of SEQ ID NO: 2 or an amino acid sequence with at least 80% identity to SEQ ID NO: 2, and
c. Valg2 comprising or consisting of the amino acid sequence of SEQ ID NO: 3 or an amino acid sequence with at least 80% identity to SEQ ID NO: 3.

In a presently preferred embodiment, the genetically modified cell capable of producing a sialylated HMO, which comprises a recombinant nucleic acid sequence encoding an enzyme with α-2,6-sialyltransferase activity as described herein is capable of producing LST-c in an amount of at least 10% of the total molar HMO content produced by the cell.

In some embodiments the genetically engineered cell is capable of producing a mixture of 6′SL and LST-c, preferably the cell produces a ratio of 6′SL:LST-c between 1:1 and 4:1, such as from 1.5:1 to 3:1.

In other embodiments the genetically engineered cell is capable of producing LST-c with low amounts of 6′SL by-product, preferably the cell produces a ratio of LST-c:6′SL, preferably the cell produces a ratio of above 2:1, such as a above 3.5:1, preferably above 15:1, such as between 5:1 to 20:1. In an even more preferred embodiment the cell does not produce any 6′SL at all or below 1% of 6′SL.

The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

### Host Cells

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Propionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromonospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella phaffii, Kluyveromyces lactis, Yarrowia lipolytica, Pichia pastoris, and Saccharomyces cerevisiae or filamentous fungi such as Aspergillus sp, Fusarium sp or Trichoderma sp, exemplary species are A. niger, A. nidulans, A. oryzae, F. solani, F. graminearum and T. reesei.

In one or more exemplary embodiments, the genetically engineered cell is selected from the group consisting of E. coli, C. glutamicum, L. lactis, B. subtilis, S. lividans, P. pastoris and S. cerevisiae.

In one or more exemplary embodiments, the genetically engineered cell is B. subtilis.

In one or more exemplary embodiments, the genetically engineered cell is S. cerevisiae or P pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### a Recombinant Nucleic Acid Sequence

The present invention relates to a genetically modified cell comprising a recombinant nucleic acid sequence encoding an enzyme with α-2,6-sialyltransferase activity, such as an enzyme selected from the group consisting of Shal, HAC1268 and valg2, and wherein said cell produces Human Milk Oligosaccharides (HMO). In particular, a sialylated HMO, and preferably with a molar % content of LST-c above, or at least of 10% of the total HMO produced.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., a sialyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present invention relates to a nucleic acid construct comprising a recombinant nucleic acid sequence encoding a sialyltransferase, wherein said recombinant nucleic acid sequence is selected from the group consisting of nucleic acid sequences encoding Shal, HAC1268 and Valg2, such as SEQ ID NO: 13, 14 and 15, or functional variants thereof.

One embodiment of the invention is a nucleic acid construct comprising a recombinant nucleic acid sequence encoding a sialyltransferase, wherein said recombinant nucleic acid sequence is selected from the group consisting of a) Shal comprising or consisting of the nucleic acid sequences of SEQ ID NO: 13 or an nucleic acid sequence with at least 80%, such as at least 85%, such as at least 90%, such as at least 95%, or such as at least 99% sequence identity to SEQ ID NO: 13; b) HAC1268 comprising or consisting the nucleic acid sequences of SEQ ID NO: 14 or an nucleic acid sequence with at least 80%, such as at least 85%, such as at least 90%, such as at least 95%, or such as at least 99% sequence identity to SEQ ID NO: 14; and/or c) Valg2 comprising or consisting the nucleic acid sequence of SEQ ID NO: 15 or an nucleic acid sequence with at least 80%, such as at least 85%, such as at least 90%, such as at least 95%, or such as at least 99% sequence identity to SEQ ID NO: 15. Preferably, the sialyltransferase encoding sequence is under the control of a promoter sequence selected from promotor sequences with a nucleic acid sequence as identified in Table 2.

TABLE 2
Selected promoter sequences

| | % Activity | | | Seq |
|---|---|---|---|---|
| | relative to | | | ID in |
| Promoter name | PglpF* | Strength | Reference | appl. |
| PmglB_70UTR_SD8 | 291% | high | WO2020255054 | 28 |
| PmglB_70UTR_{—} | 233-281% | high | WO2020255054 | 29 |
| SD10 | | | | |
| PmglB_54UTR | 197% | high | WO2020255054 | 30 |
| Plac_70UTR | 182-220% | high | WO2019123324 | 31 |
| PmglB_70UTR_SD9 | 180-226% | high | WO2020255054 | 32 |
| PmglB_70UTR_SD4 | 153%-353% | high | WO2020255054 | 33 |
| PmglB_70UTR_SD5 | 146-152% | high | WO2020255054 | 34 |
| PglpF_SD4 | 140-161% | high | WO2019123324 | 35 |
| PmglB_70UTR_SD7 | 127-173% | high | WO2019123324 | 36 |
| PmglB_70UTR | 124-234% | high | WO2020255054 | 37 |
| PglpA_70UTR | 102-179% | high | WO2019123324 | 38 |
| PglpT_70UTR | 102-240% | high | WO2019123324 | 39 |
| PglpF | 100% | high | WO2019123324 | 40 |
| PglpF_SD10 | 88-96% | high | WO2019123324 | 41 |
| PglpF_SD5 | 82-91% | high | WO2019123324 | 42 |
| PglpF_SD8 | 81-82% | high | WO2019123324 | 43 |
| PmglB_16UTR | 78-171% | high | WO2019123324 | 44 |
| PglpF_SD9 | 73-93% | middle | WO2019123324 | 45 |
| PglpF_SD7 | 47-57% | middle | WO2019123324 | 46 |
| PglpF_SD6 | 46-47% | middle | WO2019123324 | 47 |
| PglpA_16UTR | 38-64% | middle | WO2019123324 | 48 |
| Plac_wt* | 15-28% | low | WO2019123324 | 49 |
| PglpF_SD3 | 9% | low | WO2019123324 | 50 |
| PglpF_SD1 | 5% | low | WO2019123324 | 51 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically modified cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assay is performed at 30° C. Samples are preheated, the assay initiated by addition of 200 μl ortho-nitro-phenyl-β-galactosidase (4 mg/ml) and stopped by addition of 500 μl of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

In embodiments the expression of said nucleic acid sequences of the present invention is under control of a PglpF (SEQ ID NO: 40) or Plac (SEQ ID NO: 49) promoter or PmglB_UTR70 (SEQ ID NO: 37) or PglpA_70UTR (SEQ ID NO: 38) or PglpT_70UTR (SEQ ID NO: 39) or variants thereof such as promoters identified in Table 2, in particular the PglpF variant of SEQ ID NO: 35 or Plac variant of SEQ ID NO: 31, or PmglB_70UTR variants of SEQ ID NO: 28, 29, 32, 33, 34, 36 and 37. Further suitable variants of PglpF, PglpA_70UTR, PglpT_70UTR and PmglB_70UTR promoter sequences are described in or WO2019/123324 and WO2020/255054 respectively (hereby incorporated by reference).

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage λ or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NOs: 13, 14 and 15 [nucleic acid encoding Shal, HAC1268 or Valg2, respectively].

In particular, the present disclosure relates to one or more of a recombinant nucleic acid sequence and/or to a functional homologue thereof having a sequence which is at least 70% identical to SEQ ID NOs: 13, 14 or 15 [nucleic acids encoding Shal, HAC1268 or Valg2, respectively], such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of 30 BLOSUM62) substitution matrix. The output of Needle labelled “identity” (obtained using the -nobrief option) is used as the percent identity. Generally sequence identity may be calculated as follows: (Identical Residues×100)/(Length of Aligned region).

For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labelled “identity” (obtained using the -nobrief option) is used as the percent identity. Generally sequence identity may be calculated as follows: (Identical Deoxyribonucleotides×100)/(Length of Aligned region).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of table 4, should ideally be able to participate in the production of sialylated HMOs, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Modified Cell

The disclosure also relates to any commercial use of the genetically modified cell(s) or the nucleic acid construct(s) disclosed herein, such as, but not limited to, in a method for producing a sialylated human milk oligosaccharide (HMO).

In an exemplified embodiment, the genetically modified cell and/or the nucleic acid construct according to the invention is used in the manufacturing of HMOs. Preferably, in the manufacturing of HMOs, wherein the molar % content of LST-c produced by the genetically modified cell is above 10%, such as above 11% of the total HMO.

In an exemplified embodiment, the genetically modified cell and/or the nucleic acid construct according to the invention is used in the manufacturing of one or more sialylated HMO(s), wherein the sialylated HMOs are 6′SL and/or LST-c.

In an exemplified embodiment, the genetically modified cell and/or the nucleic acid construct according to the invention is used in the manufacturing of a mixture of HMO(s), comprising at least two HMOs selected from 6′SL, LNT-II, LNnT, pLNnH and LST-c.

In an exemplified embodiment, the genetically modified cell and/or the nucleic acid construct according to the invention is used in the manufacturing of a mixture of HMO(s), comprising of 6′SL, LNnT, pLNnH and/or LST-c.

In an exemplified embodiment, the genetically modified cell and/or the nucleic acid construct according to the invention is used in the manufacturing of a mixture of HMO(s), consisting of 6′SL, LNnT, pLNnH and LST-c.

In an exemplified embodiment, the genetically modified cell and/or the nucleic acid construct according to the invention is used in the manufacturing of a mixture of HMO(s), comprising 6′SL and LST-c.

In an exemplified embodiment, the genetically modified cell and/or the nucleic acid construct according to the invention is used in the manufacturing of one or more sialylated HMO(s), wherein the HMOs are 6′SL and/or LST-c.

In one or more embodiments, the genetically engineered cell and/or the nucleic acid construct is used in the manufacturing of 6′SL.

In one or more exemplary embodiments, the genetically engineered cell and/or the nucleic acid construct is used in the manufacturing of LST-c.

Production of these HMO's may require the presence of two or more glycosyltransferase activities.

### a Method for Producing Sialylated Human Milk Oligosaccharides (HMOs)

The present invention also relates to a method for producing a sialylated human milk oligosaccharide (HMO), said method comprises culturing a genetically modified cell according to the present invention.

The present invention relates to a method for producing human milk oligosaccharides (HMOs), wherein the molar % content of LST-c produced by the genetically modified cell is above 10% of the total HMO.

The present invention thus relates to a method for producing a sialylated human milk oligosaccharide (HMO), said method comprising culturing a genetically modified cell, said cell comprising:
a recombinant nucleic acid sequence encoding an enzyme with α-2,6-sialyltransferase activity, wherein said enzyme is selected from the group consisting of:
a. Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1,
b. HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, and/or
c. Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3; and wherein said cell produces a sialylated HMO.

A further embodiment of the invention is a method for producing one or more sialylated human milk oligosaccharides (HMO), said method comprising culturing a genetically modified cell comprising
a. a recombinant nucleic acid sequence encoding an enzyme with β-1,3-N-acetyl-glucosaminyltransferase activity; and
b. a recombinant nucleic acid sequence encoding an enzyme with a β-1,4-galactosyltransferase activity; and
c. a recombinant nucleic acid sequence encoding an enzyme with α-2,6-sialyltransferase activity, wherein said enzyme is selected from the group consisting of:
   i. Shal comprising or consisting of the amino acid sequence of SEQ ID NO: 1 or an amino acid sequence with at least 80% identity to SEQ ID NO: 1,
   ii. HAC1268 comprising or consisting of the amino acid sequence of SEQ ID NO: 2 or an amino acid sequence with at least 80% identity to SEQ ID NO: 2, and
   iii. Valg2 comprising or consisting of the amino acid sequence of SEQ ID NO: 3 or an amino acid sequence with at least 80% identity to SEQ ID NO: 3; or
   iv. Pmult comprising or consisting of the amino acid sequence of SEQ ID NO: 4 or an amino acid sequence with at least 80% identity to SEQ ID NO: 4; or
   v. Plst6_119 comprising or consisting of the amino acid sequence of SEQ ID NO: 5 or an amino acid sequence with at least 80% identity to SEQ ID NO: 5; and
wherein at least one of the sialylated HMOs is LST-c.

In one or more exemplary embodiments, the α-2,6-sialyltransferase of the present invention is under control of a PglpF, a Plac, or a PmglB_70UTR, a PglpA_70UTR, or a PglpT_70UTR promoter. Thus, in an exemplary embodiment, the α-2,6-sialyltransferase of the present invention is under control of a PglpF promoter or a variant thereof (table 2). In another exemplary embodiment, the α-2,6-sialyltransferase of the present invention is under control of a PmglB promoter or a variant thereof (table 2). Preferably, the recombinant nucleic acid encoding an enzyme with α-2,6-sialyltransferase is under control of a strong promoter selected from the group consisting of SEQ ID NOs: 28 to 44.

Further genetic modifications can e.g., be selected from inclusion of additional glycosyltransferases and/or metabolic pathway engineering, and inclusion of MFS transporters, as described in the above sections, which the skilled person will know how to combine into a genetically modified cell capable of producing one or more sialylated HMO's.

The method particularly comprises culturing a genetically modified cell that produces a sialylated HMO, wherein the LST-c content produced by said cell is at least 10% of the total HMO content produced by the cell. In addition, the method comprises culturing a genetically modified cell that produces a sialylated HMO.

The method comprising culturing a genetically modified cell that produces a sialylated HMO and further comprises culturing said genetically engineered cell in in the presence of an energy source (carbon source) selected from the group consisting of glucose, sucrose, fructose, xylose and glycerol.

In one aspect, the method according to the present invention produces a sialylated human milk oligosaccharide (HMO), such as 6′SL and/or LST-c.

In one aspect, the method according to the present invention produces, one or more HMO(s), wherein the HMOs are 6′SL, LNnT, pLNnH and/or LST-c.

In one aspect, the method according to the present invention, produces a mixture of HMO(s), comprising at least two HMOs, such as at least three HMOs selected from 6′SL, LNnT and LST-c.

In one aspect, the method according to the present invention produces a mixture of HMO(s), comprising at least two HMOs selected from 6′SL, LNnT and LST-c.

In one aspect, the method according to the present invention produces a mixture of HMO(s), comprising or consisting of 6′SL, LNnT, pLNnH and LST-c.

In one aspect, the method according to the present invention produces a mixture of HMO(s), comprising 6′SL and LST-c.

In one aspect, the method according to the present invention produces one or more sialylated HMO(s), wherein the HMOs are 6′SL and/or LST-c.

In one aspect, the method according to the present invention produces one or more sialylated HMO(s), wherein the HMOs are 6′SL and/or LST-c.

In one aspect, the method according to the present invention produces 6′SL.

In one aspect, the method according to the present invention produces LST-c.

To enable the production of sialylated HMOs in the method according to the present invention, the genetically modified cell may comprise a biosynthetic pathway for making a sialic acid sugar nucleotide, alternatively sialic acid can be added during cultivation of the cell.

In preferred embodiments of the methods of the present invention, the genetically modified cell comprises a biosynthetic pathway for making a sialic acid sugar nucleotide. Preferably, in methods of the present invention, the sialic acid sugar nucleotide is CMP-Neu5Ac. Thus, in methods of the present invention the sugar nucleotide pathway is expressed by the genetically modified cell, wherein the CMP-Neu5Ac pathway is encoded by the neuBCA operon from Campylobacter jejuni of SEQ ID NO: 25. In methods of the present invention, the sialic acid sugar nucleotide pathway is encoded from a high-copy plasmid bearing the neuBCA operon. In alternative embodiments the pathway for making a sialic acid sugar nucleotide is encoded from the genome of the cell, preferably by inserting heterologous DNA encoding neuA, neuB and neuC separately under control of individual promoter sequences.

The method of the present invention comprises providing a glycosyl donor, which is synthesized separately by one or more genetically engineered cells and/or is exogenously added to the culture medium from an alternative source.

In one aspect, the method of the present invention further comprises providing an acceptor saccharide as substrate for the HMO formation, the acceptor saccharide comprising at least two monosaccharide units, which is exogenously added to the culture medium and/or has been/is produced by fermentation, such as a separate microbial fermentation.

In one aspect, the method of the present invention comprises providing an acceptor saccharide comprising at least two monosaccharide units, which is exogenously added to the culture medium and/or has been/is produced by fermentation, such as a separate microbial fermentation and which is selected form lactose, LNT-II and LNnT. In a preferred embodiment the substrate for HMO formation is lactose which is fed to the culture during the fermentation of the genetically engineered cell.

The sialylated human milk oligosaccharide (HMO) is retrieved from the culture, either from the culture medium and/or the genetically modified cell. Preferably, the product oligosaccharide, e.g., LST-c, is purified to produce at least 75% pure LST-c, such as at least 85%, such as at least 95% pure LST-c.

In particular, the present invention relates to a method for producing LST-c, said method comprising:
a) obtaining a genetically modified cell comprising
   i. a recombinant nucleic acid sequence, preferably under control of a PglpF promoter, encoding an enzyme with α-2,6-sialyltransferase activity, wherein said enzyme is selected from the group consisting of: Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3, Pmult comprising or consisting of the amino acid sequence of SEQ ID NO: 4 or an amino acid sequence with at least 80% identity to SEQ ID NO: 4, and Plst6_119 comprising or consisting of the amino acid sequence of SEQ ID NO: 5 or an amino acid sequence with at least 80% identity to SEQ ID NO: 5; and
   ii. at least one nucleic acid sequence encoding a heterologous β-1,4-galactosyltransferase, such as GalT from Helicobacter pylori, and
   iii. optionally, a nuclei acid sequence encoding a β-1,3-N-acetyl-glucosaminyl-transferase, such as LgtA from Neisseria meningitidis, preferably; and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing LST-c, said method comprising:
a) obtaining a genetically modified cell comprising
   i. a recombinant nucleic acid sequence encoding an enzyme with α-2,6-sialyltransferase activity, wherein said enzyme is Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing LST-c, said method comprising:
a) obtaining a genetically modified cell comprising
   i. a recombinant nucleic acid sequence encoding an enzyme with α-2,6-sialyltransferase activity, wherein said enzyme is HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2; and
   ii. at least one nucleic acid sequence encoding a heterologous β-1,4-galactosyltransferase that is GalT from Helicobacter, iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing LST-c, said method comprising:
a) obtaining a genetically modified cell comprising
   i. a recombinant nucleic acid sequence encoding an enzyme with α-2,6-sialyltransferase activity, wherein said enzyme is Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing LST-c, said method comprising:
a) obtaining a genetically modified cell comprising
   i. a recombinant nucleic acid sequence encoding an enzyme with α-2,6-sialyltransferase activity, wherein said enzyme is pmult comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing LST-c, said method comprising:
a) obtaining a genetically modified cell comprising
   i. a recombinant nucleic acid sequence encoding an enzyme with α-2,6-sialyltransferase activity, wherein said enzyme is plst6_119 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose or LNT-II, and
c) producing said sialylated human milk oligosaccharide (HMO), in particular LST-c, by said genetically modified cell, and
d) retrieving the sialylated human milk oligosaccharide (HMO), in particular LST-c, from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is selected from the group consisting of: Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1, HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2, Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3; Pmult comprising or consisting of the amino acid sequence of SEQ ID NO: 4 or an amino acid sequence with at least 80% identity to SEQ ID NO: 4, and Plst6_119 comprising or consisting of the amino acid sequence of SEQ ID NO: 5 or an amino acid sequence with at least 80% identity to SEQ ID NO: 5, and
   ii. at least one nucleic acid sequence encoding a heterologous β-1,4-galactosyltransferase, such as GalT from Helicobacter pylori,
   iii. optionally, a nucleic acid sequence encoding a β-1,3-N-acetyl-glucosaminyl-transferase, such as LgtA from Neisseria meningitidis, and
b) culturing said genetically modified cell in a carbon-source containing culture medium and in the presence of lactose, and
c) producing said sialylated human milk oligosaccharides (HMO) 6′SL and LST-c, by said genetically modified cell, and
d) retrieving the sialylated human milk oligosaccharides (HMO) 6′SL and LST-c, from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is Shal comprising or consisting of the amino acid sequence of SEQ ID NO: 1 or an amino acid sequence with at least 80%, such as at least 85%, such as at least 90%, such as at least 95%, or such as at least 99% sequence identity to SEQ ID NO: 1 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose, and
c) producing said sialylated human milk oligosaccharides (HMO) 6′SL and LST-c, by said genetically modified cell, and
d) retrieving the sialylated human milk oligosaccharides (HMO) 6′SL and LST-c, from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is HAC1268 comprising or consisting of the amino acid sequence of SEQ ID NO: 2 or an amino acid sequence with at least 80%, such as at least 85%, such as at least 90%, such as at least 95%, or such as at least 99% sequence identity to SEQ ID NO: 2; and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis, and
b) culturing said genetically modified cell in a carbon-source containing culture medium and in the presence of lactose, and
c) producing said sialylated human milk oligosaccharides (HMO) 6′SL and LST-c, by said genetically modified cell, and
d) retrieving the sialylated human milk oligosaccharides (HMO) 6′SL and LST-c from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is Valg2 comprising or consisting of the amino acid sequence of SEQ ID NO: 3 or an amino acid sequence with at least 80%, such as at least 85%, such as at least 90%, such as at least 95%, or such as at least 99% sequence identity to SEQ ID NO: 3 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis and
b) culturing said genetically modified cell in a carbon-source containing culture medium and in the presence of lactose, and
c) producing said sialylated human milk oligosaccharides (HMOs) 6′SL and LST-c, by said genetically modified cell, and
d) retrieving the sialylated human milk oligosaccharides (HMOs) 6′SL and LST-c, from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is pmult comprising or consisting of the amino acid sequence of SEQ ID NO: 4 or an amino acid sequence with at least 80%, such as at least 85%, such as at least 90%, such as at least 95%, or such as at least 99% sequence identity to SEQ ID NO: 4 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis and
b) culturing said genetically modified cell in a carbon-source containing culture medium and in the presence of lactose, and
c) producing said sialylated human milk oligosaccharides (HMOs) 6′SL and LST-c, by said genetically modified cell, and
d) retrieving the sialylated human milk oligosaccharides (HMOs) 6′SL and LST-c, from the culture medium and/or the genetically modified cell.

In particular, the present invention relates to a method for producing 6′SL and LST-c, said method comprising:
a) obtaining a genetically modified cell comprising
   i. a recombinant nucleic acid sequence encoding an enzyme with α-2,6-sialyltransferase activity, wherein said enzyme is plst6_119 comprising or consisting of the amino acid sequence of SEQ ID NO: 5 or an amino acid sequence with at least 80%, such as at least 85%, such as at least 90%, such as at least 95%, or such as at least 99% sequence identity to SEQ ID NO: 5 and
   ii. at least one nucleic acid sequence encoding a heterologous β-1,4-galactosyltransferase that is GalT from Helicobacter pylori,
   iii. at least one a nucleic acid sequence encoding a β-1,3-N-acetyl-glucosaminyl-transferase, that is LgtA from Neisseria meningitidis and
b) culturing said genetically modified cell in a carbon-source containing culture medium and in the presence of lactose, and
c) producing said sialylated human milk oligosaccharides (HMOs) 6′SL and LST-c, by said genetically modified cell, and
d) retrieving the sialylated human milk oligosaccharides (HMOs) 6′SL and LST-c, from the culture medium and/or the genetically modified cell.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The terms “manufacturing” or “manufacturing scale” or “large-scale production” or “large-scale fermentation”, are used interchangeably and in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

At the end of culturing, the oligosaccharide as product can be accumulated both in the intra- and the extracellular matrix of the cell as well as exported into the culture medium.

The method according to the present invention comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this invention, the sialylated HMO produced can be collected from the cell culture or fermentation broth in a conventional manner.

### Retrieving/Harvesting

The sialylated human milk oligosaccharide (HMO) is retrieved from the culture medium and/or the genetically modified cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/182965 or WO2017/152918, wherein the latter describes purification of sialylated HMOs. In embodiments the LST-c is purified to produce at least 75% pure LST-c, such as at least 80% pure LST-c, such as at least 85% pure LST-c, such as at least 90% pure LST-c, such as at least 95% pure LST-c. The purified HMOs can be used as nutritional products, such as early life nutritional products, dietary supplements or other nutraceuticals, pharmaceuticals, or for any other purpose, e.g., for research.

### Manufactured Product

The term “manufactured product” according to the use of the genetically engineered cell or the nucleic acid construct refer to the one or more HMOs intended as the one or more product HMO(s). The various products are described above.

Accordingly, the manufactured product may be a mixture of HMOs comprising or consisting essentially of LST-c, LNnT, 6′SL and pLNnH. Accordingly, in embodiments, LST-c is in the range of 10 to 45 molar %, such as from 10-30 molar %, such as 10, 11, 12, 13, 14, 15, 16, 17, 18, 19, 20, 21, 22, 23, 24, 25, 26, 27, 28, 29, 30, 31, 32, 33, 34, 35, 36, 37, 28, 39, 40, 41, 42, 43, 44 or 45 molar % of the mixture, LNnT is in the range of 25-70 molar %, such as 40-70 molar %, such as 25, 25, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 molar % of the mixture, 6′SL is in the range of 0 to 65 molar %, such as 0-30 molar %, such as in the range of 0.1-25 molar % such as 0, 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 molar % of the mixture, and pLNnH is in the range of 1-20 molar %, such as 3-20 molar %, such as 3, 4, 5, 6, 7, 8, 9, 10, 11, 12, 13, 14, 15, 16, 17, 18, 19 or 20 molar % of the mixture.

In one embodiment the mixture of HMOs comprises or consists essentially of LST-c is in the range of 15-45 molar %, of the mixture, LNnT is in the range of 25-75 molar % of the mixture, 6′SL is in the range of 0 to 8 molar of the mixture, and pLNnH is in the range of 1-20 molar %, of the mixture.

In another embodiment the mixture of HMOs comprises or consists essentially of LST-c is in the range of 15-30 molar %, of the mixture, LNnT is in the range of 25-65 molar % of the mixture, 6′SL is in the range of 15 to 65 molar of the mixture, and pLNnH is in the range of 1-10 molar %, of the mixture.

In further embodiments the mixture is produced according to the methods of the invention and where the HMO mixture is purified such that it contains less than 15% (w/w) lactose, such as less than 10% (w/w) lactose, such as less than 5% (w/w) lactose, such as less than 2% (w/w) lactose.

Advantageously, the methods disclosed herein provide both a decreased ratio of by-product to product and an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Sequences

The current application contains a sequence listing in text format and electronical format which is hereby incorporated by reference.

An overview of the SEQ ID NOs used in the present application can be found in table 1 (alpha-2,6-sialyltransferase protein sequences), table 2 (promoter sequences) and table 4 (alpha-2,6-sialyltransferase DNA sequences), additional sequences described in the application is the DNA sequence encoding the neuBCA operon from Campylobacter jejuni (SEQ ID NO: 25) and the β-1,3-N-acetylglucosaminyltransferase LgtA from N. meningitidis (SEQ ID NO: 26), β-1,4-galactosyltransferases galT from H. pylori (SEQ ID NO: 27) and DNA sequences encoding codon optimized individual members of the neuBCA operon from Campylobacter jejuni, namely neuA (SEQ ID NO: 52), neuB (SEQ ID NO: 53) and neuC (SEQ ID NO: 54).

### Items

Various embodiments of present disclosure are described in the following items.
1. A genetically modified cell comprising a recombinant nucleic acid sequence encoding an enzyme with α-2,6-sialyltransferase activity, wherein said cell is capable of producing one or more HMO(s) and wherein at least 10% of the total molar HMO content produced by said cell is LST-c.
2. The genetically modified cell according to embodiment 1, wherein said α-2,6-sialyltransferase enzyme is selected from the group consisting of:
   a. Shal comprising or consisting of the amino acid sequence of SEQ ID NO: 1 or an amino acid sequence with at least 80% identity to SEQ ID NO: 1,
   b. HAC1268 comprising or consisting of the amino acid sequence of SEQ ID NO: 2 or an amino acid sequence with at least 80% identity to SEQ ID NO: 2, and
   c. Valg2 comprising or consisting of the amino acid sequence of SEQ ID NO: 3 or an amino acid sequence with at least 80% identity to SEQ ID NO: 3.
3. The genetically modified cell according to any one of embodiments 1 or 2, wherein the cell produces LST-c and 6′SL when lactose is used as initial substrate for the HMO formation.
4. The genetically modified cell according to any of the preceding embodiments, wherein the recombinant nucleic acid sequence encoding the α-2,6-sialyltransferase enzyme is present in more than one copy, such as two copies, three copies, four copies.
5. The genetically modified cell according to any of the preceding embodiments, wherein the wherein the recombinant nucleic acid sequence(s) encoding the α-2,6-sialyltransferase enzyme is(are) integrated into the genome of the genetically modified cell.
6. The genetically modified cell according to any of the preceding embodiments, wherein the α-2,6-sialyltransferase enzyme is Valg2 or Shal and the 6′SL:LST-c ratio is between 1:1 and 3:1.
7. The genetically modified cell according to any of the preceding embodiments, wherein the α-2,6-sialyltransferase enzyme is HAC1268 and the LST-c:6′SL ratio is above 2:1, preferably above 3.5:1, preferably above 15:1.
8. The genetically modified cell according to any of the preceding embodiments, wherein the sialyltransferase is under the control of a promoter selected from the group consisting of PglpF, Plac, PmglB_70UTR, PglpA_70UTR and PglpT_70UTR (SEQ ID NOs: 40, 49, 37, 38 and 39, respectively) and variants thereof, and wherein the promoter is preferably a strong promoter selected from the group consisting of SEQ ID NOs 28, 29, 30, 31, 32, 33, 34, 35, 36, 37, 38, 39, 40, 41, 42, 43 and 44.
9. The genetically modified cell according to any one of the preceding embodiments, wherein the cell further comprises a recombinant nucleic acid sequence encoding a β-1,4-galactosyltransferase.
10. The genetically modified cell according to embodiment 9, wherein the genetically modified cell further comprises a recombinant nucleic acid sequence encoding a β-1,3-N-acetyl-glucosaminyltransferase.
11. The genetically modified cell according to any one of embodiments 9 or 10, wherein the β-1,3-N-acetylglucosaminyltransferase is LgtA from Neisseria meningitidis (SEQ ID NO: 26) and the β-1,4-galactosyltransferase is GalT from Helicobacter pylori (SEQ ID NO: 27).
12. The genetically modified cell according to any one of the preceding embodiments, wherein the cell comprises a biosynthetic pathway for making a sialic acid sugar nucleotide.
13. The genetically modified cell according to embodiment 12, wherein the sialic acid sugar nucleotide is CMP-Neu5Ac and said pathway for making a sialic acid sugar nucleotide is encoded by the nucleic acid sequence encoding neuBCA from Campylobacter jejuni (SEQ ID NO: 25).
14. The genetically modified cell according to embodiment 12 or 13, wherein the sialic acid sugar nucleotide pathway is encoded from a high-copy plasmid bearing the neuBCA operon.
15. The genetically modified cell according to embodiment 12 to 14, wherein the pathway for making a sialic acid sugar nucleotide is encoded from genomically integrated neuA, neuB and neuC, wherein the three genes preferably are under control of individual promoters.
16. The genetically modified cell according to any of the preceding embodiments, wherein said modified cell is a microorganism.
17. The genetically modified cell according to any of the preceding embodiments, wherein said modified cell is a bacterium or a fungus.
18. The genetically modified cell according to embodiment 17, wherein said fungus is selected from a yeast cell of the genera Komagataella, Kluyveromyces, Yarrowia, Pichia, Saccharomyces, Schizosaccharomyces or Hansenula or from a filamentous fungus of the genera Aspergillus, Fusarium or Trichoderma.
19. The genetically modified cell according to embodiment 17, wherein said bacterium is selected from the group consisting of Escherichia sp., Bacillus sp., lactobacillus sp., Corynebacterium sp. and Campylobacter sp.
20. The genetically modified cell according to embodiment 19 wherein said bacterium is E. coli.
21. A method for producing one or more sialylated human milk oligosaccharides (HMO), said method comprising culturing a genetically modified cell comprising,
   a. a recombinant nucleic acid sequence encoding an enzyme with β-1,3-N-acetyl-glucosaminyltransferase activity; and
   b. a recombinant nucleic acid sequence encoding an enzyme with a β-1,4-galactosyltransferase activity; and
   c. a recombinant nucleic acid sequence encoding an enzyme with α-2,6-sialyltransferase activity, wherein said enzyme is selected from the group consisting of:
      i. Shal comprising or consisting of the amino acid sequence of SEQ ID NO: 1 or an amino acid sequence with at least 80% identity to SEQ ID NO: 1,
      ii. HAC1268 comprising or consisting of the amino acid sequence of SEQ ID NO: 2 or an amino acid sequence with at least 80% identity to SEQ ID NO: 2,
      iii. Valg2 comprising or consisting of the amino acid sequence of SEQ ID NO: 3 or an amino acid sequence with at least 80% identity to SEQ ID NO: 3;
      iv. Pmult comprising or consisting of the amino acid sequence of SEQ ID NO: 4 or an amino acid sequence with at least 80% identity to SEQ ID NO: 4; and
      v. Plst6_119 comprising or consisting of the amino acid sequence of SEQ ID NO: 5 or an amino acid sequence with at least 80% identity to SEQ ID NO: 5, and wherein at least one of the sialylated HMOs is LST-c.
22. The method according to embodiment 21, wherein said genetically modified cell comprises at least one additional modification according to any one of embodiments 8 to 14
23. The method according to embodiment 21 or 0, wherein at least 10% of the total molar HMO content produced by said method is LST-c.
24. The method according to any one of embodiments 21 to 22, where the genetically modified cell is a microorganism according to any one of embodiments 17 to 20.
25. The method according to any one of embodiments 21 to 24, wherein the sialylated human milk oligosaccharide(s) (HMO(s)) produced is LST-c and optionally 6′SL.
26. The method according to any one of embodiments 21 to 25, wherein the α-2,6-sialyltransferase enzyme is Valg2 or Shal and the 6′SL:LST-c ratio is between 1:1 and 3:1.
27. The method according to any one of embodiments 21 to 25, wherein the α-2,6-sialyltransferase enzyme is HAC1268 and the LST-c:6′SL ratio is above 2:1, preferably above 3.5:1, preferably above 15:1.
28. The method according to embodiment 21 to 27, wherein the 6′SL content produced by said cell is below 50% of the total HMO content produced by the cell.
29. The method according to any one of embodiments 21 to 25, wherein the method comprises cultivating the genetically engineered cell in the presence of an energy source selected from the group consisting of glucose, sucrose, fructose, xylose and glycerol.
30. The method according to any one of embodiments 21 to 28, wherein lactose is added during the cultivation of the genetically engineered cells as a substrate for the HMO formation.
31. The method according to any one of embodiments 21 to 28, wherein LNT-II is added during the cultivation of the genetically engineered cells as a substrate for the HMO formation.
32. The method according to any one of embodiments 21 to 31 wherein the sialylated human milk oligosaccharide(s) (HMO(s)) is retrieved from the culture medium and/or the genetically modified cell.
33. The method according to claim 32, wherein the LST-c is purified to produce at least 75% pure LST-c, such as at least 85%, such as at least 95% pure LST-c.
34. A nucleic acid construct comprising recombinant nucleic acid sequence encoding an enzyme with α-2,6-sialyltransferase activity, wherein said recombinant nucleic acid sequence is selected from the group consisting of:
   a. Shal comprising or consisting of the nucleic acid sequences of SEQ ID NO: 13 or a nucleic acid sequence with at least 80% identity to SEQ ID NO: 13,
   b. HAC1268 comprising or consisting of the nucleic acid sequences of SEQ ID NO: 14 or a nucleic acid sequence with at least 80% identity to SEQ ID: 14, and/or
   c. Valg2 comprising or consisting of the nucleic acid sequence of SEQ ID NO: 15 or a nucleic acid sequence with at least 80% identity to SEQ ID: 15; and
   wherein the enzyme encoding sequence is under the control of a promoter sequence selected from the group consisting of PglpF, Plac, PmglB_70UTR, PglpA_70UTR and PglpT_70UTR (SEQ ID NOs: 40, 49, 37, 38 and 39, respectively) and variants thereof.
35. A nucleic acid construct comprising a recombinant nucleic acid sequence encoding an enzyme with α-2,6-sialyltransferase activity according to embodiment 34, for use in a host cell for producing a sialylated HMO, wherein at least 10% of the total molar HMO content produced by the method is LST-c.
36. A genetically modified cell according to any one of embodiments 1 to 20, for use in the production of at least one sialylated HMO.
37. A mixture of HMOs comprising essentially of LST-c, LNnT, 6′SL and pLNnH.
38. The mixture of HMOs according to embodiment 36, wherein
   a. LST-c is in the range of 10-45 molar % of the mixture,
   b. LNnT is in the range of 25-70 molar % of the mixture,
   c. 6′SL is in the range of 0-65 molar % of the mixture, and
   d. pLNnH is in the range of 1-20 molar % of the mixture.
39. The mixture of HMOs according to embodiment 36 or 37, wherein
   a. LST-c is in the range of 15-45 molar % of the mixture,
   b. LNnT is in the range of 25-75 molar % of the mixture,
   c. 6′SL is in the range of 0-8 molar % of the mixture, and
   d. pLNnH is in the range of 1-20 molar % of the mixture.
40. The mixture of HMOs according to embodiment 36 or 37, wherein
   a. LST-c is in the range of 10-30 molar % of the mixture,
   b. LNnT is in the range of 25-65 molar % of the mixture,
   c. 6′SL is in the range of 15-65 molar % of the mixture, and
   d. pLNnH is in the range of 1-10 molar % of the mixture.
41. The mixture of HMOs according to embodiment 36 to 40, wherein the mixture is produced according to the methods of embodiments 21 to 33 and where the HMO mixture is purified such that it contains less than 15% (w/w) lactose.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes:

19 enzymes were collected following an in-silico selection approach that was based on protein BLAST searches using known α-2,6-sialyltransferases as queries and by exploiting information sources such as scientific articles or databases, e.g., the KEGG and CAZY databases.

TABLE 3
List of the enzymes tested in the framework of the present invention

| Enzyme | | |
|---|---|---|
| Name | GenBank ID* | Origin |
| Shal | WP_108944675.1 | Shewanella halifaxensis |
| HAC1268 | STP03859.1 (2 point mut) | Helicobacter acinonychis str. Sheeba |
| valg2 | WP_169629213.1 | Vibrio alginolyticus |
| pmult | WP_005753497.1 | Pasteurela multocida |
| plst6_119 | BAI49484.1 | Photobacterium leiognathi JT-SHIZ-119 |
| ppho1 | WP_065208987.1 | Photobacterium phosphoreum |
| pdam | WP_151182185.1 | Photobacterium damselae |
| pst6-224 | BAF92026.1 | Photobacterium sp JT-ISH-224 |
| plst6_145 | BAF91416.1 | Photobacterium leiognathi JT-SHIZ-145 |
| Pd2 | BAA25316.1 | Photobacterium damsela JT0160 |
| phot | WP_036792497.1 | Photobacterium MULTISPECIES |
| Poral_2 | WP_101774701.1 | Pasteurella oralis |
| Alstel | WP_063457849.1 | Alteromonas stellipolaris |
| Flavoba | MBL20167.1 | Flavobacteriaceae bacterium |
| ppho2 | WP_107198328.1 | Photobacterium phosphoreum |
| Gene11 | WP_067376979.1 | Marinobacterium aestuarii |
| Drome | AAG13185.1 | Drosophila melanogaster |
| Celter1 | MBD5788313.1 | Cellulosimicrobium terreum |
| PM70 | AAK03258.1 | Pasteurella multocida subsp. multocida str. Pm70 |

*the sequences used in the present application may be truncated at the N- or C-terminal as compared to the GenBank sequence.

### Strains

The strains (genetically engineered cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

This MDO strain was further engineered to generate an LNnT producing strain by chromosomally integrating a beta-1,3-GlcNAc transferase (LgtA from Neisseria meningitidis, homologous to NCBI Accession nr. WP_033911473.1 and shown as SEQ ID NO: 26) and a beta-1,4-galactosyltransferase (GalT from Helicobacter pylori, homologous to GenBank ID WP_001262061.1 and shown as SEQ ID NO: 27) both under the control of a PglpF promoter (SEQ ID NO: 40), this strain is named the LNnT strain.

Codon optimized DNA sequences encoding individual α-2,6-sialyltransferases were genomically integrated into the LNnT strain. Additionally, each strain was transformed with a high-copy plasmid bearing the neuBCA operon from Campylobacter jejuni (SEQ ID NO: 25) under the control of the Plac promoter. The neuBCA operon encodes all the enzymes required for the formation of an activated sialic acid sugar nucleotide (CMP-Neu5Ac). CMP-Neu5Ac acts as a donor for the intended glycosyltransferase reaction facilitated by the α-2,6-sialyltransferase under investigation, i.e., the transfer of sialic acid from the activated sugar CMP-Neu5Ac to the terminal galactose of LNnT (acceptor) to form LST-c.

The genotypes of the background strain (MDO), LNnT strain and the α-2,6-sialyltransferase-expressing strains capable of producing LST-c are provided in Table 4.

TABLE 4
Genotypes of the strains, capable of producing
LST-c, used in the present examples.

| | | 2,6-ST |
|---|---|---|
| | | cDNA |
| Strain | | SEQ |
| ref | Genotype | ID NO |
| MDO | F-λ- ΔendA1 ΔrecA1 ΔrelA1 ΔgyrA96 Δthi-1 | — |
| | glnV44 hsdR17(rK-mK-) ΔlacZ wcaF::Plac | |
| | ΔnanKETA ΔlacA ΔmelA ΔwcaJ ΔmdoH | |
| LNnT | MDO, 2x IgtA-PglpF, 1xgalT-PglpF | — |
| Shal | LNnT, Shal-PglpF, pBS-neuBCA(Plac)-amp | 13 |
| HAC1268 | LNnT, HAC1268-PglpF, pBS-neuBCA(Plac)-amp | 14 |
| Valg2 | LNnT, Valg2-PglpF, pBS-neuBCA(Plac)-amp | 15 |
| Pmult | LNnT, Pmult -PglpF, pBS-neuBCA(Plac)-amp | 16 |
| Plst6_119 | LNnT, Plst6_119-PglpF, pBS-neuBCA(Plac)-amp | 17 |
| Ppho1 | LNnT, Ppho1-PglpF, pBS-neuBCA(Plac)-amp | 18 |
| Plst6_145 | LNnT, Plst6_145-PglpF, pBS-neuBCA(Plac)-amp | 19 |
| Pdam | LNnT, Pdam -PglpF, pBS-neuBCA(Plac)-amp | 20 |
| Poral_2 | LNnT, Poral_2-PglpF, pBS-neuBCA(Plac)-amp | 21 |
| Phot | LNnT, Phot-PglpF, pBS-neuBCA(Plac)-amp | 22 |
| Pd2 | LNnT, Pd2-PglpF, pBS-neuBCA(Plac)-amp | 23 |
| Pst6-224 | LNnT, Pst6_145 -PglpF, pBS-neuBCA(Plac)-amp | 24 |

*2,6ST is an abbreviation of alpha-2,6-sialyltransferase, and the sequence is inserted into the genome of the host strain.

### Deep Well Assay

The strains were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7.5) to start the main culture. The new BMM was supplemented with magnesium sulphate, thiamine, a bolus of 20% glucose solution (50 ul per 100 mL) and a bolus of 20% lactose solution (5 ml per 100 ml). Moreover, 50% sucrose solution was provided as carbon source, accompanied by the addition of sucrose hydrolase (invertase), so that glucose was released at a rate suitable for C-limited growth. IPTG (50 mg/ml) was added to induce gene expression and ampicillin antibiotic (100 mg/ml). The main cultures were incubated for 72 hours at 28° C. and 1000 rpm shaking

### Fermentation

The E. coli strains were cultivated in 2 replicates carried out in 250 mL fermenters (Ambr 250 Bioreactor system, Sartorius) starting with 100 mL of mineral culture medium consisting of 25 g/L glucose, lactose, (NH4)2HPO4, KH2PO4, MgSO4 x 7H2O, KOH, NaOH, citric acid, trace element solution, antifoam and thiamine. The dissolved oxygen level was kept at 20% by a cascade of first agitation and then airflow starting at 700 rpm (up to max 4500 rpm) and 1 VVM (up to max 3 VVM). The pH was kept at 6.8 by titration with an 8.5% NH40H solution. The cultivations were started with 2% (v/v) inoculums from pre-cultures grown in a similar glucose containing medium to an optical density measured at 600 nm of 2.5-5. After depletion of the glucose contained in the batch medium, a feed solution containing 49% (w/w) glucose, MgSO₄ x 7H₂O, trace metals and antifoam was fed continuously using the feed profile starting at an initial feed rate of 0.224 g glucose/h that was linearly ramped up to 0.447 g/h over 5 hours and then fed at a constant rate of 0.447 g/h. The temperature was kept at 34° C. throughout. Additional lactose was added as a bolus addition at 20 hours after feed start of 25% lactose monohydrate solution and then every 19 hours to keep lactose from being limiting. The growth and metabolic activity and state of the cells were followed by on-line measurements of reflectance and CO₂ evolution rate.

Throughout the fermentation, samples were taken in order to determine the concentration of HMOs, by-products, lactose and other minor by-products using HPLC. Total broth samples were diluted three-fold in deionized water and boiled for 20 minutes. This was followed by centrifugation at 17000 g for 3 minutes, where after the resulting supernatant was analyzed by HPLC.

### Example 1—In Vivo LST-c Synthesis

Genetically modified cells expressing individual alpha-2,6-sialyltransferase enzymes were screened for their ability to produce the sialylated HMO LST-c.

A group of 19 enzymes (table 3) were compiled for testing their ability to synthesize LST-c when introduced into a genetically modified cells that produce LNnT and activated sialic acid (CMP-Neu5Ac).

Genetically modified strains expressing the 19 individual α-2,6-sialyltransferases (table 3) were generated as described in the “Method” section. The cells were screened in a in a fed-batch deep well assay setup as described in the “Method” section. The molar content of individual HMOs produced by the strains was measured by HPLC. In addition, NMR analysis was conducted on the LST-c fraction to confirm that it indeed is LST-c.

Table 4 lists the genotype of the 12 strains that were found to produce LST-c even in very small amounts, the remaining 7 strains tested did not produce any LST-c at all.

The results of the LST-c producing cells are shown in table 7 as the fraction of the total HMO content (in percentage, %) produced by each strain.

TABLE 7
Content of individual HMO's as molar % of
total HMO (mM) content produced by each strain.

| | LST-c | 6′SL | LNnT | pLNnH |
|---|---|---|---|---|
| Poral_2 | 2.4 | 12.7 | 84.9 | 0.0 |
| phot | 3.3 | 8.5 | 77.8 | 10.4 |
| Pd2 | 4.9 | 23.3 | 64.6 | 7.3 |
| plst6_145 | 8.2 | 15.6 | 41.1 | 35.1 |
| ppho1 | 8.4 | 13.1 | 69.9 | 8.6 |
| pdam | 8.9 | 16.3 | 66.3 | 8.5 |
| plst6_119 | 10.2 | 16.1 | 66.4 | 7.3 |
| pmult | 10.5 | 8.1 | 76.1 | 5.3 |
| valg2 | 11.7 | 17.2 | 64.5 | 6.6 |
| HAC1268 | 17.9 | 4.8 | 71.5 | 5.8 |
| Shal | 21.2 | 28.5 | 46.4 | 3.9 |

No additional HMOs beyond the ones indicated in table 7 were identified in the deep well assay testing the indicated strains.

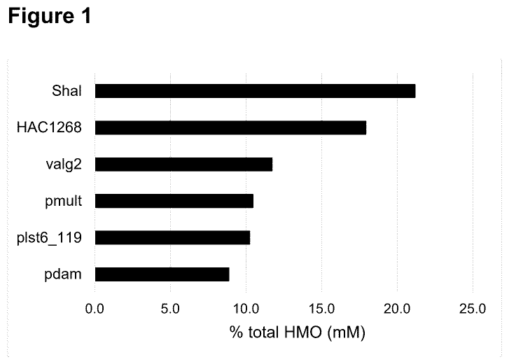

From the data presented in table 7, it can be seen that there are 5 enzymes (Shal, HAC1268, Valg2, pmult and plst6_119) that can transfer a sialic acid unit onto the terminal galactose of a LNnT molecule to form LST-c at a level above 10% of the total HMO molar content produced by each modified cell which is above the amount of LST-c produced by Pdam in this experiment. Pdam is known in the prior art and has been suggested to be active on LNnT, although WO 2021/123113 does not provide any evidence of the levels of LST-c produced. The molar % of LST-c produced by these 5 strains and the Pdam strain are shown in FIG. 1.

Furthermore, Shal, HAC1268 and Valg2 formed LST-c at a level above 10.5% of the total HMO molar content produced, which is above the level formed by plst6_119, which has previously been used in a genetically modified strain to form 6′SL, but to our knowledge has never been shown to be capable of transferring a sialic acid unit onto the terminal galactose of a LNnT molecule.

The HAC1268 produced almost 4 times more LST-c than 6′SL (which is produced by sialyation of lactose). This indicates that this enzyme has an increased activity on LNnT as substrate contrary lactose as substrate. In addition, the HAC1268 enzyme is described as a bifunctional alpha-2,3/-2,8-sialyltransferase and not an alpha-2,6-sialyltransferase, which makes it even more surprising that it is highly efficient in forming LST-c.

Expression of HAC1268 in a strain producing LNnT and sialic acid resulted in 17.9% LST-c. Similarly, expression of the Shal enzyme in a strain producing LNnT and sialic acid resulted in 21.2% LST-c, however with a significantly higher 6′SL ratio. A lower 6′SL ratio is preferred if it is desired to purify the LST-c as the separation of two acidic HMOs (LST-c and 6′SL) is more challenging than the separation of a single acidic HMO (LST-c) from the neutral HMOs (LNnT and pLNnH).

### Example 2—Fermentation Using Shal and HAC1268 α-2,6-Sialyltransferase Strains

To confirm the high level of LST-c observed in the deep well assays of Shal and HAC1268 strains of example 1, the four strains were fermented as described in the “Method” section above.

The results are shown in table 8.

TABLE 8
Content of individual HMO's as molar %
of total HMO content produced by each strain

| Strain ref. | pLNnH | LNT-II | LNnT | 6′SL | LST-c |
|---|---|---|---|---|---|
| Shal | 3.8 | 0.0 | 33.9 | 26.6 | 26.7 |
| HAC1268 | 16.8 | 0.0 | 67.2 | 0.0 | 15.4 |

No additional HMOs beyond the ones indicated in table 8 were identified in the fermentation.

From the data presented table 8, it can be seen that the fraction of LST-c for the Shal expressing strain was higher when the culturing was done in fermenters compared to the deep well assays presented in example 1, showing the ability of Shal-expressing cell to produce LST-c at a level above 26% of the total HMO produced by this strain. In fermentation HAC1268-expressing cells provided very similar LST-c levels to the deep well assays however a great benefit was the surprising absence of 6′SL in the final mixture, which makes it easier to purify the LST-c, since the only charged HMO is LST-c.

### Example 3

In the present example it was investigated if changes in the CMP-Neu5Ac production system in the strain as well as additional copies of the alpha-2,6-sialyltransferase enzymes for the Shal, HAC1268 and Valg2 strains would improve the LST-c production and change the ratio of LST-c:6′SL.

The strains were constructed by inserting the neuA (SEQ ID NO: 52), neuB (SEQ ID NO: 53) and neuC (SEQ ID NO: 54) genes under control of the PglpF promoter (SEQ ID NO: 40) into the genome at separate genomic integration sites to generate a strain capable of producing CMP-Neu5Ac from the genome instead of from a plasmid.

The genotype of the strains tested are shown in table 9. MP0 is used as background strain of the present example and MP1 to MP8 all have the same genetic background as MP0 with the additional modifications indicated in the table,

TABLE 9
Genotype of strains used in the current example

| Strain | | | 2,6-ST cDNA |
|---|---|---|---|
| Ref | Description | Genotype | SEQ ID NO |
| MP0 | LNnT and genomic | MDO, 2x IgtA-PglpF, 1xgalT-PglpF, 1xneuA-PglpF, | — |
| | CMP-Neu5Ac | 1xneuB-PglpF, 1xneuC-PglpF | |
| MP1 | Shal | MP0, 1xShal-PglpF, | 13 |
| MP2 | Shal + plasmid | MP0, 1xShal-PglpF, pBS-neuBCA(Plac)-amp | 13 |
| | CMP-Neu5Ac | | |
| MP3 | 2xShal + plasmid | MP0, 2xShal-PglpF, pBS-neuBCA(Plac)-amp | 13 |
| | CMP-Neu5Ac | | |
| MP4 | HAC1268 | MP0, 1xHAC1268-PglpF, | 14 |
| MP5 | HAC1268 + plasmid | MP0, 1xHAC1268-PglpF, pBS-neuBCA(Plac)-amp | 14 |
| | CMP-Neu5Ac | | |
| MP6 | 2xHAC1268 + plasmid | MP0, 2xHAC1268-PglpF, pBS-neuBCA(Plac)-amp | 14 |
| | CMP-Neu5Ac | | |
| MP7 | Valg2 | MP0, 1xValg2-PglpF, | 15 |
| MP8 | Valg2 + plasmid | MP0, 1xValg2-PglpF, pBS-neuBCA(Plac)-amp | 15 |
| | CMP-Neu5Ac | | |

2x indicate that two copies of the nucleic acid sequence encoding the indicated enzyme is present on the genome

### Deep Well Assay for Product Formation

The strains were screened in 96 deep well plates using a 3-day protocol for assessment of HMO production. During the first 24 hours, fresh precultures were grown to high densities. More specifically, fresh precultures were prepared using a basal minimal medium supplemented with magnesium sulphate, thiamine and glucose. The pH was adjusted to pH 7.0 with NaOH. The precultures were incubated for 24 hours at 34° C. and 1000 rpm shaking.

For HMO production an aliquot of the pre-culture was transferred to a new deep well plate with basal minimal medium (BMM7, pH 7.5) to start the main culture. The new BMM was supplemented with thiamine and magnesium sulphate, 0.01% of glucose, 2% lactose, 1.75% of Maltodextrin, thiamine, and Glucoamylase for optimized hydrolysis of maltodextrin. The HMO main cultures were incubated for 48 hours at 28° C. and 1000 rpm shaking. The assay was generally performed in triplicates.

After incubation of the main cultures were boiled and centrifuged. Samples were analyzed by HPLC.

### Results

The strains from table 9 were tested in the deep well assay described in relation to this example. Table 10 shows the average level from at least 3 repeats pr strain of the following HMOs, LNT-II, LNnT, pLNnH, 6′SL, LST-c. In addition, an LST-c isomer was identified which essentially is an LNnT molecule with the sialic acid at a different moiety than the terminal galactose indicating that some of alpha-2,6-sialyltransferase enzymes have some affinity for other moieties in LNnT.

The results of the LST-c producing cells are shown in table 10 as the fraction of the total HMO+LST-c isomer content (in percentage, %) produced by each strain.

TABLE 10
Content of individual HMO's/isomer as molar % of total
content of the listed molecules produced by each strain

| | | | | | | | LST-c |
|---|---|---|---|---|---|---|---|
| Strain | Description | LNT-II | LNnT | pLNnH | 6′SL | LST-c | isomer |
| MP1 | x1 Shal | 1.1 | 45.6 | 4.7 | 28.0 | 19.9 | 1.3 |
| MP2 | x1 Shal + extra | 1.1 | 34.9 | 2.2 | 53.9 | 23.9 | 0.4 |
| | CMP-Sia | | | | | | |
| MP3 | x2 Shal + extra | 1.0 | 30.6 | 0.8 | 61.8 | 19.3 | 1.5 |
| | CMP-Sia | | | | | | |
| MP4 | x1 HAC1268 | 1.1 | 53.5 | 6.7 | 1.2 | 24.2 | 0.2 |
| MP5 | x1 HAC1268 + | 1.7 | 48.5 | 4.5 | 1.7 | 29.2 | 0.1 |
| | extra CMP-Sia | | | | | | |
| MP6 | x2 HAC1268 + | 1.1 | 44.1 | 2.5 | 5.0 | 39.4 | 0.1 |
| | extra CMP-Sia | | | | | | |
| MP7 | x1 Valg2 | 0.6 | 50.3 | 5.7 | 30.6 | 16.7 | 3.3 |
| MP8 | x1 Valg2 + extra | 0.5 | 39.7 | 3.6 | 49.4 | 19.8 | 3.5 |
| | CMP-Sia | | | | | | |

From these data it can be seen that exchanging the CMP-Neu5Ac formation from a plasmid-based system (example 1) to a genomically integrated system (current example) still resulted in efficient sialyation of LNnT for all three enzymes. Since, the analysis is conducted with a modified deep well assay as compared to example 1, the data are however not directly comparable.

What can be observed for all three enzymes, based on table 10, is that when introduced into a strain that contains both a genomically integrated CMP-Neu5Ac production pathway and the CMP-Neu5Ac production pathway on a plasmid, the formation of LST-c increases, in that MP2 produces 4% more LST-c than MP1, MP5 produces 5% more LST-c than MP4 and MP8 produces 3% more LST-c than MP7. For the Shal and Valg2 the increased formation of CMP-Neu5Ac however also significantly increases the amount of 6′SL formed leading to an LST-c:6′SL ratio of 0.44 and 0.40, respectively, whereas the LST-c:6′SL ratio for HAC1268 is 17.2. The high LST-c:6′SL ratio is a significant benefit if pure LST-c is desired, since it is more difficult to separate 6′SL and LST-c than any of the neutral HMOs from LST-c.

A further benefit of HAC1268 seems to be that this enzyme produces very little of the LST-c isomer compared to Shal and Valg2. In particular, it is worth noting that the production of the isomer does not increase in the HAC1268 strain even when the copy number of the nucleic acid sequence encoding the enzyme is increased to two (MP6), whereas for Shal there is an increase in the LST-c Isomer when an additional copy of the nucleic acid sequence encoding the enzyme is added to the high producing CMP-Neu5Ac strain (MP3 vs MP2).

With respect to the insertion of an additional copy of the nucleic acid sequence encoding the alpha-2,6-sialyltransferase this provides a further increase of LST-c production in the HAC1268 strain of another 10% to a total of 39% (MP6), whereas for the Shal strain the increase in enzyme activity seems to result in an increased 6′SL formation and a reduction of LST-c (MP3). This clearly indicates that HAC1268 has a very high specificity towards the terminal galactose moiety on LNnT, and low specificity towards both lactose and other moieties in LNnT, which makes it an ideal enzyme for the production of pure LST-c.

If the formation of a mixture of 6′SL and LST-c is desired Shal and valg2 seems to be highly suitable enzymes and here the ratio of 6′SL and LST-c can be adjusted by regulating either the enzyme activity (e.g. by increasing the copy number of the nucleic acid sequence encoding the enzyme) or by regulating the CMP-Neu5Ac formation. With the three Shal strains and the two valg2 strains tested the ratio of 6′SL:LST-c can be in the range from 1.5:1 to 3:1.

1. A genetically modified cell comprising a recombinant nucleic acid sequence encoding an enzyme with α-2,6-sialyltransferase activity, wherein the cell is capable of producing one or more HMO(s) and wherein at least 10% of the total molar HMO content produced by the cell is LST-c.

2. The genetically modified cell according to claim 1, wherein the α-2,6-sialyltransferase enzyme is selected from the group consisting of:
a. HAC1268 comprising or consisting of the amino acid sequence of SEQ ID NO: 2 or an amino acid sequence with at least 8-090% identity to SEQ ID NO: 2,
b. Shal comprising or consisting of the amino acid sequence of SEQ ID NO: 1 or an amino acid sequence with at least 8090% identity to SEQ ID NO: 1, and
c. Valg2 comprising or consisting of the amino acid sequence of SEQ ID NO: 3 or an amino acid sequence with at least 908-0% identity to SEQ ID NO: 3.

3. The genetically modified cell according to claim 1, wherein the cell further comprises a recombinant nucleic acid sequence encoding a β-1,4-galactosyltransferase.

4. The genetically modified cell according to claim 3, wherein the cell further comprises a recombinant nucleic acid sequence encoding a β-1,3-N-acetyl-glucosaminyltransferase.

5. The genetically modified cell according to claim 1, wherein the cell further comprises a biosynthetic pathway for making a sialic acid sugar nucleotide.

6. The genetically modified cell according to claim 1, wherein the modified cell is a bacterium or a fungus.

7. The genetically modified cell according to claim 6, wherein the fungus is selected from a yeast cell of the genera Komagataella, Kluyveromyces, Yarrowia, Pichia, Saccharomyces, Schizosaccharomyces or Hansenula or from a filamentous fungus of the genera Aspergillus, Fusarium or Trichoderma, or the bacterium is selected from the group consisting of Escherichia sp., Bacillus sp., lactobacillus sp., Corynebacterium sp. and Campylobacter sp.

8. The genetically modified cell according to claim 6, wherein the bacterium is E. coli.

9. A method for producing at least one sialylated human milk oligosaccharides (HMO), the method comprising culturing a genetically modified cell comprising,
a. a recombinant nucleic acid sequence encoding an enzyme with α-2,6-sialyltransferase activity, wherein said enzyme is selected from the group consisting of:
i. HAC1268 comprising or consisting of the amino acid sequence of SEQ ID NO: 2 or an amino acid sequence with at least 90% identity to SEQ ID NO: 2,
ii. Shal comprising or consisting of the amino acid sequence of SEQ ID NO: 1 or an amino acid sequence with at least 90% identity to SEQ ID NO: 1,
iii. Valg2 comprising or consisting of the amino acid sequence of SEQ ID NO: 3 or an amino acid sequence with at least 90% identity to SEQ ID NO: 3;
iv. Pmult comprising or consisting of the amino acid sequence of SEQ ID NO: 4 or an amino acid sequence with at least 90% identity to SEQ ID NO: 4; and
v. Plst6_119 comprising or consisting of the amino acid sequence of SEQ ID NO: 5 or an amino acid sequence with at least 90% identity to SEQ ID NO: 5, and
b. a recombinant nucleic acid sequence encoding an enzyme with a β-1,4-galactosyltransferase activity;
wherein at least one of the sialylated HMOs is LST-c.

10. The method according to claim 9, wherein the genetically modified cell further comprises a recombinant nucleic acid sequence encoding an enzyme with β-1,3-N-acetyl-glucosaminyltransferase activity.

11. The method according to claim 9, wherein at least 10% of the total molar HMO content produced by the method is LST-c.

12. The method according to claim 9, wherein the at least one sialylated HMO comprises LST-c and 6′SL.

13. The method according to claim 9, wherein the culturing step comprises providing lactose or LNT-II as a substrate for the HMO formation.

14. The method according to claim 9, further comprising retrieving the at least one sialylated HMO from a culture medium and/or the genetically modified cell.

15. The method according to claim 14, further comprising purifying the LST-c to at least 75% pure LST-c.

16.-18. (canceled)

19. A mixture of HMOs consisting essentially of LST-c, LNnT, 6′SL and pLNnH.

20. The mixture of HMOs according to claim 19, wherein
LST-c is in the range of 10-45 molar % of the mixture,
LNnT is in the range of 25-70 molar % of the mixture,
6′SL is in the range of 0-65 molar % of the mixture, and
pLNnH is in the range of 1-20 molar % of the mixture.

21. The mixture of HMOs according to claim 20, wherein the mixture contains less than 15% (w/w) lactose.

22. The method according to claim 9, wherein the genetically modified cell is a bacterium or a fungus and the genetically modified cell further comprises a recombinant nucleic acid sequence encoding a β-1,3-N-acetyl-glucosaminyltransferase and/or a biosynthetic pathway for making a sialic acid sugar nucleotide.
